(19) United States
(12) Patent Application Publication
(10) Pub. No.: US 2014/0086959 A1
(43) Pub. Date: Mar. 27, 2014

(54) METHOD FOR PRODUCING MILK HAVING AN IMMUNOREGULATORY ACTION

(71) Applicant: Morinaga Milk Industry Co., Ltd., Tokyo (JP)

(72) Inventors: Takahiro Ochiya, Tokyo (JP); Nobuyoshi Kosaka, Tokyo (JP); Kazunori Sekine, Zama-shi (JP); Hirohisa Izumi, Zama-shi (JP)

(73) Assignee: Morinaga Milk Industry Co., Ltd., Tokyo (JP)

(21) Appl. No.: 14/066,456

(22) Filed: Oct. 29, 2013

Related U.S. Application Data
(62) Division of application No. 13/322,127, filed on Nov. 22, 2011.

(30) Foreign Application Priority Data
Jul. 14, 2009 (JP) 2009-165991

(51) Int. Cl. C12N 15/113

(52) U.S. Cl. CPC C12N 15/113
USPC 424/278.1

(57) ABSTRACT

A method for screening for a foodstuff providing production of milk having an immunoregulatory action, a foodstuff having an immunoregulatory action, and a method for producing it are provided. A diet or substance that increases or decreases an amount of microRNA present in milk of a mammal is identified by using correlation of microRNA profiles in the milk and a diet ingested by the mammal or a substance contained in the diet as an index to screen for a diet or a substance providing production of breast milk having an immunoregulatory action.

### CROSS-REFERENCE TO RELATED APPLICATIONS

This application is a divisional of U.S. application Ser. No. 13/322,127, filed Nov. 22, 2011 which is the U.S. National Phase under 35 U.S.C. §371 of International Application PCT/JP2010/061926, filed Jul. 14, 2010, which was published in a non-English language, which claims priority to JP Patent Application No. 2009-165991, filed Jul. 14, 2009. The above applications are incorporated herein by reference.

### TECHNICAL FIELD

The present invention relates to a method for screening for a diet providing production of milk having an immunoregulatory action, which is useful in the fields of foodstuff, animal feed, and so forth.

### BACKGROUND ART

Immunity of living organisms essentially functions for the purpose of “defense” against external attacks. For example, phylaxis and elimination of cancer cells correspond to the “defense”, and enhancement of the immunity effectively operates in such a case.

On the other hand, overresponse of the immunity, i.e., “hyperimmunity”, may adversely affect living organisms. Examples thereof include allergic responses, autoimmune diseases, chronic inflammations, and so forth. It is known that, in such a case, symptoms are improved by suppressing production of inflammatory cytokines such as IL-6, TNF-α and IL-1.

Further, it is becoming clear that immunostimulating actions functioning for the purpose of “defense” against external attacks, and immunosuppressive actions functioning for suppressing allergic responses, autoimmune diseases, chronic inflammations etc. induced by hyperimmunoreaction are regulated by microRNA (henceforth also referred to as “miRNA”).

After a miRNA is transcribed from genome, it undergoes two times of cleavage and becomes a non-coding small RNA of about 22 bases. It is known that, as a function thereof, it binds to a 3′-untranslated region of target mRNAs in a sequence-complementary manner to suppress translation of the target mRNAs. One kind of miRNA inhibits translation of a plurality of kinds of mRNAs in a cell to regulate various functions of the cell. Many reports have been made especially on relations thereof with development and evolution of cancers, and relation between miRNA and diseases attracts attention.

For example, as for miR-181, it has been reported that it is involved in development of B cells, activation of T cells, and development of immunity (Non-patent documents 1 to 3).

As for miR-155, it is known that it is involved in development of immunity through activation of the innate immunity (Non-patent documents 1 and 4) and regulation of differentiation and functions of T cells and B cells (Non-patent documents 1 and 5), and it is involved in antiallergy and anti-inflammation through regulation of Th1/Th2 balance (Non-patent documents 1 and 6) and maintenance of the functions of regulatory T cells, which suppress hyperimmunoreactions (Non-patent document 7).

miR-17 and miR-92 cooperate to regulate differentiation and development of B cells and T cells and thereby participate in development of immunity (Non-patent documents 1, 8 and 9).

It is known that miR-223 participates in phylaxis by controlling proliferation and activation of neutrophils (Non-patent documents 1 and 10), miR-150 participates in phylaxis by suppressing differentiation of B cells (Non-patent document 1 and 11), and let-7i participates in phylaxis by controlling TLR4 expression in cholangiocytes (Non-patent document 12).

It is known that miR-125 participates in anti-inflammation by suppressing production of TNF-α (Non-patent documents 1 and 13).

It is known that miR-146 participates in phylaxis by negatively regulating the innate immunity (Non-patent documents 1 and 14), and participates in antiallergy by controlling Th1/Th2 balance (Non-patent document 15).

It has recently been reported that miRNAs which function in cells as translation regulatory molecules are present in a lipid bilayer called exosome, and are secreted out of the cells (Non-patent document 16). Since it has also been confirmed that secreted miRNAs are incorporated into other cells, presence of intercellular actions by means of miRNA has been presented. Further, exosomes are known to be present in various kinds of human body fluids. In particular, presence of miRNAs in human plasma and serum has already been reported, and a possibility of use thereof as a biomarker of prostate cancer or uterine cancer has been suggested (Non-patent document 17).

Body fluids containing exosomes include, besides plasma and serum, saliva, urine, amniotic fluid and breast milk (Non-patent document 17). Among these, breast milk is a body fluid produced by mammals in a specific period, and responsible for transfer of substances between individuals, i.e., from a mother to a child. Moreover, breast milk not only supplements nutrients to a child, but also gives immune substances acquired by a mother to a child.

Breast milk contains secretory IgA, lactoferrin, lysozyme, cytokines, and so forth, and it is considered that it protects infants from infection, and promotes development of infant's immunity (Non-patent document 19). Actually, it is known that children grown up on breast milk involve a lower risk of infection in the bronchi or intestinal tract as compared to children not grown in such a manner. Breast milk contains IgA, lactoferrin, glycoproteins, glycolipids etc. which show antibacterial activities, as well as cytokines which regulate immunocytes. However, the objects analyzed in the researches to date are mainly proteins contained in breast milk, and although there are reports on nucleic acids contained in breast milk, researches on nucleic acids contained in breast milk and having specific sequences have not been reported.

Moreover, it is also known that development of mammary glandular cells controlled by expression of cyclooxygenase 2 is regulated by miR-101a (Non-patent document 20). However, it is not suggested that miRNAs exist in milk.

In addition, after the priority date of this application, it has been reported that microRNAs are present in microvesicles derived from bovine milk (Patent document 21), and microRNAs are identified in fresh milk of bovines of different lactation periods, commercial liquid milk and dried milk (Patent document 22).

### PRIOR ART REFERENCES

### Non-Patent Documents

Non-patent document 1: Lindsay, M. A., Trends Immunol, 29:343-351, 2008
Non-patent document 2: Li, Qi-Jing et al., Cell, 129:147-161, 2007
Non-patent document 3: Chen, Chang-Zheng et al., Science, 303:83-86, 2004
Non-patent document 4: O'Connel, R. M. et al., PNAS, 104 (5):1604-1609, 2007
Non-patent document 5: Vigorito, E. et al., Immunity, 27:847-859, 2007
Non-patent document 6: Rodriguez, A. et al., Science, 316:608-611, 2007
Non-patent document 7: Kohlhaas, S. et al., J. Immunol., 182:2578-2582, 2009
Non-patent document 8: Koralov, S. B. et al., Cell, 132:860-874, 2008
Non-patent document 9: Xiao, C. et al., Nat. Immunol., 9:405-414, 2008
Non-patent document 10: Jonathan, B. et al., Nature, 451:1125-1129, 2008
Non-patent document 11: Zhou, B. et al., PNAS, 104 (17):7080-7085, 2007
Non-patent document 12: Chen, Xian-Ming et al., J. Biol. Chem., 282 (39):28929-28938, 2007
Non-patent document 13: Tili, E. et al., J. Immunol., 179:5082-5089, 2007
Non-patent document 14: Taganov, K. D. et al., PNAS, 103 (33):12481-12486, 2006
Non-patent document 15: Monticelli, S. et al., Genome Biol., 6, R71, 2005
Non-patent document 16: Valadi, H. et al., Nat. Cell Biol., 9:654-659, 2007
Non-patent document 17: Gilad, S. et al., PLoS One, 3 (9):e3148, 2008
Non-patent document 18: Admyre, C., J. Immunol., 179:1969-1978, 2007
Non-patent document 19: Goldman, A. S., Breastfeed Med., 2 (4):195-204, 2007
Non-patent document 20: Tanaka, T. et al., Differentiation, 77:181-187, 2009
Non-patent document 21: Hata, T. et al., Biochem. Biophys. Res. Commun., 396 (2):528-533, 2010
Non-patent document 22: Chen, X. et al., Cell Research, (2010):1-10

### SUMMARY OF THE INVENTION

### Object to be Achieved by the Invention

An object of the present invention is to provide a method for screening for a diet providing production of milk having an immunoregulatory action, a novel foodstuff having an immunoregulatory action, and a method for producing it.

### Means for Achieving the Object

The inventors of the present invention conducted researches with paying attention to the fact that breast milk affected maturation of infant's immune system. As a result, they found that immunity-related miRNAs are highly expressed in breast milk, and accomplished the present invention.

The present invention thus provides a method for screening for a diet or a substance providing production of breast milk having an immunoregulatory action, which comprises identifying a diet or a substance that increases or decreases amount of microRNA present in milk of a mammal by using correlation of microRNA profile in the milk and a diet ingested by the mammal or a substance contained in the diet as an index.

In an embodiment of the aforementioned method, the immunoregulatory action is an immunostimulating action, and when the amount of the microRNA increases, it is judged that the diet or substance provides production of breast milk having an immunostimulating action.

In a preferred embodiment of the aforementioned method, microRNA profiles in the milk observed before and after ingestion of the diet are compared, and when amount of at least one kind of microRNA observed after the ingestion is higher than that observed before the ingestion, it is judged that the diet increases the amount of the microRNA in the milk.

In another preferred embodiment of the aforementioned method, microRNA profiles in the milk and microRNA profiles in serum or plasma are compared, and when amount of microRNA contained in both the milk and the serum or plasma is increased in the milk by ingestion of the diet in a degree of 1.2 times or more as compared to that observed in the serum or plasma, it is judged that the diet increases the amount of the microRNA in the milk.

In another embodiment of the aforementioned method, the immunoregulatory action is an immunosuppressive action, and when the amount of the microRNA decreases, it is judged that the diet or substance provides production of breast milk having an immunosuppressive action.

In a preferred embodiment of the aforementioned method, microRNA profiles in the milk observed before and after the ingestion of the diet are compared, and when the amount of at least one kind of microRNA observed after the ingestion is lower than that observed before the ingestion, it is judged that the diet decreases the amount of the microRNA in the milk.

In a preferred embodiment of the aforementioned method, microRNA profiles in the milk and microRNA profiles in serum or plasma are compared, and when amount of microRNA contained in both the milk and the serum or plasma is decreased in the milk by ingestion of the diet in a degree of 0.8 times or less of that observed in the serum or plasma, it is judged that the diet decreases the amount of the microRNA in the milk.

In a preferred embodiment of the aforementioned method, the mammal is a human.

In a preferred embodiment of the aforementioned method, the microRNA profiles consists of amount of microRNA selected from the group consisting of miR-10, miR-15, miR-16, miR-17, miR-18, miR-19, miR-20, miR-21, miR-22, miR-23, miR-24, miR-25, miR-26, miR-27, miR-28, miR-29, miR-30, miR-31, miR-33, miR-34, miR-92, miR-93, miR-96, miR-98, miR-99, miR-100, miR-101, miR-103, miR-106, miR-107, miR-125, miR-126, miR-128, miR-129, miR-130, miR-133, miR-134, miR-139, miR-140, miR-141, miR-143, miR-146, miR-148, miR-151, miR-152, miR-155, miR-181, miR-182, miR-183, miR-184, miR-185, miR-186, miR-188, miR-192, miR-193, miR-195, miR-196, miR-199, miR-200, miR-203, miR-204, miR-205, miR-206, miR-210, miR-212, miR-214, miR-218, miR-219, miR-221, miR-222, miR-223, miR-290, miR-291, miR-292, miR-294, miR-296, miR-301, miR-320, miR-322, miR-324, miR-327, miR-328, miR-331, miR-338, miR-340, miR-341, miR-342, miR-345, miR-347, miR-352, miR-361, miR-362, miR-365, miR-370, miR-375, miR-378, miR-409, miR-425, miR-429, miR-452, miR-455, miR-465, miR-466, miR-483, miR-484, miR-486, miR-494, miR-497, miR-500, miR-503, miR-532, miR-542, miR-584, miR-652, miR-664, miR-672, miR-685, miR-708, miR-760, miR-872, miR-874, miR-877, miR-1224, miR-1300, miR-1307, let-7a, let-7b, let-7c, let-7d, le-7e, let-7f, and let-7i.

In a preferred embodiment of the aforementioned method, the microRNA profiles consists of amount of microRNA selected from the group consisting of miR-15, miR-16, miR-17, miR-18, miR-19, miR-20, miR-21, miR-23, miR-24, miR-26, miR-27, miR-29, miR-30, miR-33, miR-34, miR-92, miR-93, miR-99, miR-100, miR-101, miR-106, miR-107, miR-125, miR-130, miR-140, miR-141, miR-143, miR-146, miR-155, miR-181, miR-185, miR-186, miR-192, miR-193, miR-195, miR-200, miR-205, miR-210, miR-218, miR-219, miR-221, miR-222, miR-223, miR-301, miR-322, miR-340, miR-361, miR-370, miR-429, miR-455, miR-466, miR-497, miR-500, miR-503, miR-532, miR-542, let-7d, and let-7i.

In a preferred embodiment of the aforementioned method, the microRNA profiles consists of amount of microRNA selected from the group consisting of miR-15, miR-16, miR-19, miR-21, miR-23, miR-24, miR-26, miR-27, miR-30, miR-34, miR-99, miR-106, miR-107, miR-125, miR-130, miR-140, miR-181, miR-193, miR-210, miR-222, miR-223, miR-361, miR-370, miR-429, miR-500, miR-532, let-7d, and let-7i.

The present invention also provides a method for producing milk or dairy products having an immunoregulatory action, which comprises the step of giving a diet or a substance identified to increase or decrease amount of microRNA in milk of a mammal by the aforementioned screening method to a mammal (except for human), and the step of collecting milk of the mammal.

In an embodiment of the aforementioned method, the immunoregulatory action is an immunostimulating action, and the diet or substance is identified to increase the amount of the microRNA.

In an embodiment of the aforementioned method, the immunoregulatory action is an immunosuppressive action, and the diet or substance is identified to decrease the amount of the microRNA.

The present invention also provides a composition for oral ingestion having an immunostimulating action, which comprises a base for a composition for oral ingestion and microRNA added to the base.

In a preferred embodiment of the composition for oral ingestion, the microRNA is selected from the group consisting of miR-10, miR-15, miR-16, miR-17, miR-18, miR-19, miR-20, miR-21, miR-22, miR-23, miR-24, miR-25, miR-26, miR-27, miR-28, miR-29, miR-30, miR-31, miR-33, miR-34, miR-92, miR-93, miR-96, miR-98, miR-99, miR-100, miR-101, miR-103, miR-106, miR-107, miR-125, miR-126, miR-128, miR-129, miR-130, miR-133, miR-134, miR-139, miR-140, miR-141, miR-143, miR-146, miR-148, miR-151, miR-152, miR-155, miR-181, miR-182, miR-183, miR-184, miR-185, miR-186, miR-188, miR-192, miR-193, miR-195, miR-196, miR-199, miR-200, miR-203, miR-204, miR-205, miR-206, miR-210, miR-212, miR-214, miR-218, miR-219, miR-221, miR-222, miR-223, miR-290, miR-291, miR-292, miR-294, miR-296, miR-301, miR-320, miR-322, miR-324, miR-327, miR-328, miR-331, miR-338, miR-340, miR-341, miR-342, miR-345, miR-347, miR-352, miR-361, miR-362, miR-365, miR-370, miR-375, miR-378, miR-409, miR-425, miR-429, miR-452, miR-455, miR-465, miR-466, miR-483, miR-484, miR-486, miR-494, miR-497, miR-500, miR-503, miR-532, miR-542, miR-584, miR-652, miR-664, miR-672, miR-685, miR-708, miR-760, miR-872, miR-874, miR-877, miR-1224, miR-1300, miR-1307, let-7a, let-7b, let-7c, let-7d, le-7e, let-7f, and let-7i.

In a preferred embodiment of the composition for oral ingestion, the microRNA is selected from the group consisting of miR-15, miR-16, miR-17, miR-18, miR-19, miR-20, miR-21, miR-23, miR-24, miR-26, miR-27, miR-29, miR-30, miR-33, miR-34, miR-92, miR-93, miR-99, miR-100, miR-101, miR-106, miR-107, miR-125, miR-130, miR-140, miR-141, miR-143, miR-146, miR-155, miR-181, miR-185, miR-186, miR-192, miR-193, miR-195, miR-200, miR-205, miR-210, miR-218, miR-219, miR-221, miR-222, miR-223, miR-301, miR-322, miR-340, miR-361, miR-370, miR-429, miR-455, miR-466, miR-497, miR-500, miR-503, miR-532, miR-542, let-7d, and let-7i.

In a preferred embodiment of the composition for oral ingestion, the microRNA is selected from the group consisting of miR-15, miR-16, miR-19, miR-21, miR-23, miR-24, miR-26, miR-27, miR-30, miR-34, miR-99, miR-106, miR-107, miR-125, miR-130, miR-140, miR-181, miR-193, miR-210, miR-222, miR-223, miR-361, miR-370, miR-429, miR-500, miR-532, let-7d, and let-7i.

In a preferred embodiment of the composition for oral ingestion, the composition is a foodstuff for infants or a foodstuff for little children.

In a preferred embodiment of the composition for oral ingestion, the foodstuff for infants or the foodstuff for little children is infant formula or follow-up formula.

### BRIEF DESCRIPTION OF THE DRAWINGS

FIG. 1 shows results of detection of miRNAs in human breast milk obtained by microarray analysis.

FIG. 2 shows comparison of miR-181a levels in breast milk for first six months after birth and six months thereafter. hsa represents human, and cel represents a nematode (Caenorhabditis elegans) (the same shall apply to the following drawings).

FIG. 3 shows comparison of miR-155, miR-17, and miR-92 levels in breast milk for first six months after birth and six months thereafter.

FIG. 4 shows comparison of immunity-related miRNA levels in human breast milk and serum.

FIG. 5 shows comparison of miRNA levels observed before and after freeze-thaw.

FIG. 6 shows comparison of miRNA levels observed before and after storage at low pH (pH 1).

FIG. 7 shows comparison of miRNA levels observed after RNases treatment and without RNases treatment.

### EMBODIMENTS FOR CARRYING OUT THE INVENTION

The method of the present invention is a method for screening for a diet or a substance providing production of breast milk having an immunoregulatory action, which comprises identifying a diet or a substance that increases or decreases amount of microRNA present in milk of a mammal on the basis of correlation of microRNA profiles in the milk and a diet ingested by the mammal or a substance contained in the diet.

In an embodiment of the aforementioned method of the present invention, the immunoregulatory action is an immunostimulating action, and when the amount of the microRNA increases, it is judged that the diet or substance provides production of breast milk having an immunostimulating action. In another embodiment of the aforementioned method of the present invention, the immunoregulatory action is an immunosuppressive action, and when the amount of the microRNA decreases, it is judged that the diet or substance provides production of breast milk having an immunosuppressive action.

The present invention is based on a concept that an immunoregulatory action is expected to be obtained by oral administration of miRNA, because of the novel finding that miRNAs are contained in milk, and the fact that miRNAs can stably exist even under acidic conditions in the stomach, and breast milk promotes development of immunity in infants ingesting the breast milk (for example, Breastfeed Med., 2(4):195-204, 2007). And, on the basis of a prediction that a miRNA profile in milk is affected by diet, it was thought to identify a diet or an active ingredient contained in it that could increase or decrease amount of miRNA present in milk.

The immunoregulatory action defined for the screening method, milk, dairy product, and so forth of the present invention includes, for example, both an action of enhancing immunopotentiating action, which functions for the purpose of “defense” against external attacks (immunostimulating action), and an immunosuppressive action suppressively functioning against overresponse by the immunity, i.e., allergic responses, autoimmune diseases, chronic inflammations etc., in which “hyperimmunoreaction” adversely affect living organisms.

The terms “immunostimulating action” and “immunosuppressive action” are used in a relative meaning. When an immunopotentiating action usually observed for breast milk of a certain mammal is enhanced after ingestion of the diet or substance, the breast milk has an immunostimulating action, and when the immunopotentiating action is decreased, the breast milk has an immunosuppressive action. When the immunopotentiating action observed after ingestion of the diet or substance by a mammal is enhanced as compared to that observed before the ingestion, the breast milk of the mammal has an immunostimulating action, and when the immunopotentiating action is decreased as compared to that observed before the ingestion, the breast milk has an immunosuppressive action.

The correlation of miRNA profiles in milk of a mammal and a diet ingested by the mammal or a substance contained in the diet can be investigated, for example, as follows.

Milk is collected from a mammal that ingested a diet, and a miRNA profile in the milk is examined.

The mammal is not particularly limited, and examples include human, bovine, goat, ovine, swine, ape, dog, cat, rat, mouse, hamster, guinea pig, and so forth. The mammal is preferably human or bovine.

In the present invention, the miRNA profile consists of type and amount of miRNA. The miRNA may consist of one kind of miRNA, or two or more kinds of miRNAs. Type of miRNA is not particularly limited, so long as those existing in milk are chosen, and examples include miR-10, miR-15, miR-16, miR-17, miR-18, miR-19, miR-20, miR-21, miR-22, miR-23, miR-24, miR-25, miR-26, miR-27, miR-28, miR-29, miR-30, miR-31, miR-33, miR-34, miR-92, miR-93, miR-96, miR-98, miR-99, miR-100, miR-101, miR-103, miR-106, miR-107, miR-125, miR-126, miR-128, miR-129, miR-130, miR-133, miR-134, miR-139, miR-140, miR-141, miR-143, miR-146, miR-148, miR-151, miR-152, miR-155, miR-181, miR-182, miR-183, miR-184, miR-185, miR-186, miR-188, miR-192, miR-193, miR-195, miR-196, miR-199, miR-200, miR-203, miR-204, miR-205, miR-206, miR-210, miR-212, miR-214, miR-218, miR-219, miR-221, miR-222, miR-223, miR-290, miR-291, miR-292, miR-294, miR-296, miR-301, miR-320, miR-322, miR-324, miR-327, miR-328, miR-331, miR-338, miR-340, miR-341, miR-342, miR-345, miR-347, miR-352, miR-361, miR-362, miR-365, miR-370, miR-375, miR-378, miR-409, miR-425, miR-429, miR-452, miR-455, miR-465, miR-466, miR-483, miR-484, miR-486, miR-494, miR-497, miR-500, miR-503, miR-532, miR-542, miR-584, miR-652, miR-664, miR-672, miR-685, miR-708, miR-760, miR-872, miR-874, miR-877, miR-1224, miR-1300, miR-1307, let-7a, let-7b, let-7c, let-7d, le-7e, let-7f, let-7i, and the like.

These miRNAs are those of which presence is confirmed in either one of human breast milk, colostrum of rat, or colostrum of bovine. As described above, it is known that breast milk promotes development of immunity in infants who ingest it (for example, Breastfeed Med., 2(4):195-204, 2007). Moreover, it has been reported that many components considered to be important to the immune system of infants (including animal infants) are generally contained in colostrum (J. Anim. Sci., 2009, 87:(Suppl. 1): 3-9). Therefore, it is suggested that the aforementioned miRNAs of which presence in milk is confirmed are involved in immune functions.

Among those mentioned above, preferred are miR-15, miR-16, miR-17, miR-18, miR-19, miR-20, miR-21, miR-23, miR-24, miR-26, miR-27, miR-29, miR-30, miR-33, miR-34, miR-92, miR-93, miR-99, miR-100, miR-101, miR-106, miR-107, miR-125, miR-130, miR-140, miR-141, miR-143, miR-146, miR-155, miR-181, miR-185, miR-186, miR-192, miR-193, miR-195, miR-200, miR-205, miR-210, miR-218, miR-219, miR-221, miR-222, miR-223, miR-301, miR-322, miR-340, miR-361, miR-370, miR-429, miR-455, miR-466, miR-497, miR-500, miR-503, miR-532, miR-542, let-7d, and let-7i. These are miRNAs for which immunoregulatory action is reported, miRNAs of which presence in colostrum of both of rat and bovine was confirmed, or miRNAs of which amount increased in colostrum of rat administered with Bifidobacterium bacteria.

Moreover, among the miRNAs mentioned above, particularly preferred are miR-15, miR-16, miR-19, miR-21, miR-23, miR-24, miR-26, miR-27, miR-30, miR-34, miR-99, miR-106, miR-107, miR-125, miR-130, miR-140, miR-181, miR-193, miR-210, miR-222, miR-223, miR-361, miR-370, miR-429, miR-500, miR-532, let-7d, and let-7i. These are miRNAs of which presence was confirmed in colostrum of both rat and bovine.

Certain miRNAs have subtypes, and for example, 2 to 4 kinds of subtypes are known for each of miR-181, miR-92, miR-125, miR-146, and so forth, such as miR-181a, miR-181b, miR-181c, miR-181d, miR-92a, miR-92b, miR-125a, miR-125a-3P, miR-125a-5P, miR-125b, miR-146a, miR-146b, miR-146b-3P and miR-146b-5P, respectively. Certain other miRNAs also have subtypes, and in the present invention, the miRNA may be any of such subtypes. Examples of the subtypes include those of which presence in milk was confirmed in the examples described later (refer to Examples 1, 3, 4 and 5).

The nucleotide sequences of human miR-155 precursor, hsa-mir-155 (MI0000681), and the active site thereof, hsa-miR-155 (MIMAT0009241), are shown in SEQ ID NOS: 1 and 2, respectively. Shown in the parentheses are accession numbers in a miRNA database (miRBase::Sequences, http://microrna.sanger.ac.uk/sequences/index.shtml) (the same shall apply to the following descriptions).

The nucleotide sequences of bovine miR-155 precursor, bta-miR-155 (MI0009752), and the active site thereof, bta-miR-155 (MIMAT0000646), are shown in SEQ ID NOS: 3 and 4, respectively.

The nucleotide sequences of human miR-181a precursors, hsa-mir-181a-1 (MI0000289), and hsa-mir-181a-2 (MI0000269), and the active site thereof, hsa-miR-181a (MIMAT0000256), are shown in SEQ ID NOS: 5, 6 and 7, respectively.

The nucleotide sequences of human miR-181b precursors, hsa-mir-181b-1 (MI0000270), and hsa-mir-181b-2 (MI0000683), and the active site thereof, hsa-miR-181b (MIMAT0000257), are shown in SEQ ID NOS: 8, 9 and 10, respectively.

The nucleotide sequences of bovine miR-181a precursors, bta-mir-181a (MI0004757), and bta-mir-181a-1 (MI0010484), and the active site thereof, bta-miR-181a (MIMAT0003543), are shown in SEQ ID NOS: 11, 12 and 13, respectively.

The nucleotide sequences of bovine miR-181b precursors, bta-mir-181b-1 (MI0010485), and bta-mir-181b-2 (MI0005013), and the active site thereof, bta-miR-181b (MIMAT0003793), are shown in SEQ ID NOS: 14, 15 and 16, respectively.

The nucleotide sequences of human miR-223 precursor, hsa-mir-223 (MI0000300), and the active site thereof, hsa-miR-223 (MIMAT0000280), are shown in SEQ ID NOS: 17 and 18, respectively.

The nucleotide sequences of bovine miR-223 precursor, bta-mir-223 (MI0009782), and the active site thereof, bta-miR-223 (MIMAT0009270), are shown in SEQ ID NOS: 19 and 20, respectively.

The nucleotide sequences of human miR-17 precursor, hsa-mir-17 (MI0000071), and the active site thereof, hsa-miR-17 (MIMAT0000070) (also called hsa-miR-17-5p), are shown in SEQ ID NOS: 21 and 22, respectively.

The nucleotide sequences of bovine miR-17 precursor, bta-mir-17 (MI0005031), the active sites thereof, bta-miR-17-5p (MIMAT0003815) and bta-miR-17-3p (MIMAT0003816), are shown in SEQ ID NOS: 23, 24 and 25, respectively.

The nucleotide sequences of human miR-92a precursors, hsa-mir-92a-1 (MI0000093), and hsa-mir-92a-2 (MI0000094), and the active site thereof, hsa-miR-92a (MIMAT0000092), are shown in SEQ ID NOS: 26, 27 and 28, respectively.

The nucleotide sequences of human miR-92b precursor, hsa-mir-92b (MI0003560), and the active site thereof, hsa-miR-92b (MIMAT0003218), are shown in SEQ ID NOS: 29 and 30, respectively.

The nucleotide sequences of bovine miR-92 precursor, bta-mir-92 (MI0005024), and the active site thereof, bta-miR-92 (MIMAT0003808), are shown in SEQ ID NOS: 31 and 32, respectively.

The nucleotide sequences of bovine miR-92a precursor, bta-mir-92a (MI0009905), and the active site thereof, bta-miR-92a (MIMAT0009383), are shown in SEQ ID NOS: 33 and 34, respectively.

The nucleotide sequences of bovine miR-92b precursor, bta-mir-92b (MI0009906), and the active site thereof, bta-miR-92b (MIMAT0009384), are shown in SEQ ID NOS: 35 and 36, respectively.

The nucleotide sequences of human let-7i precursor, hsa-let-7i (MI0000434), and the active site thereof, hsa-let-7i (MIMAT0000415), are shown in SEQ ID NOS: 37 and 38, respectively.

The nucleotide sequences of bovine let-7i precursor, bta-let-7i (MI0005065), and the active site thereof, bta-let-7i (MIMAT0003851), are shown in SEQ ID NOS: 39 and 40, respectively.

The nucleotide sequences of human miR-125a precursor, hsa-mir-125a (MI0000469), and the active sites thereof, hsa-miR-125a-5p (MIMAT0000443) and hsa-miR-125a-3p (MIMAT0004602), are shown in SEQ ID NOS: 41, 42 and 43, respectively.

The nucleotide sequences of human miR-125b precursors, hsa-mir-125b-1 (MI0000446), and hsa-mir-125b-2 (MI0000470), and the active site thereof, hsa-miR-125b (MIMAT0000423), are shown in SEQ ID NOS: 44, 45 and 46, respectively.

The nucleotide sequences of bovine miR-125a precursor, bta-mir-125a (MI0004752), and the active site thereof, bta-miR-125a (MIMAT0003538), are shown in SEQ ID NOS: 47 and 48, respectively.

The nucleotide sequences of bovine miR-125b precursors, bta-mir-125b-1 (MI0004753), and bta-mir-125b-2 (MI0005457), and the active site thereof, bta-miR-125b (MIMAT0003539), are shown in SEQ ID NOS: 49, 50 and 51, respectively.

The nucleotide sequences of human miR-146a precursor, hsa-mir-146a (MI0000477), and the active site thereof, hsa-miR-146a (MIMAT0000449), are shown in SEQ ID NOS: 52 and 53, respectively.

The nucleotide sequences of human miR-146b precursor, hsa-mir-146b (MI0003129), and the active sites thereof, hsa-miR-146b-5p (MIMAT0002809) (also referred to as hsa-miR-146b) and hsa-miR-146b-3p (MIMAT0004766), are shown in SEQ ID NOS: 54, 55 and 56, respectively.

The nucleotide sequences of bovine miR-146a precursor, bta-mir-146a (MI0009746), and the active site thereof, bta-miR-146a (MIMAT0009236), are shown in SEQ ID NOS: 57 and 58, respectively.

The nucleotide sequences of bovine miR-146b precursor, bta-mir-146b (MI0009745), and the active site thereof, bta-miR-146b (MIMAT0009235), are shown in SEQ ID NOS: 59 and 60, respectively.

The nucleotide sequences of human miR-150 precursor, hsa-mir-150 (MI0000479), and the active site thereof, hsa-miR-150 (MIMAT0000451), are shown in SEQ ID NOS: 61 and 62, respectively.

The nucleotide sequences of bovine miR-150 precursor, bta-mir-150 (MI0005058), and the active site thereof, bta-miR-150 (MIMAT0003845), are shown in SEQ ID NOS: 63 and 64, respectively.

In addition to the aforementioned miRNAs, miRNAs of which presence in milk of rat or bovine was confirmed, and miRNAs of other animals corresponding to those miRNAs are shown as Tables 1 to 10.

TABLE 1

| | Human or | | SEQ |
|---|---|---|---|
| miRNA | animal | Sequence | ID NO |
| miR-155 | human | uuaaugcuaaucgugauaggggu | 1 |
| | bovine | uuaaugcuaaucgugauaggggu | 4 |
| miR-17-3p | bovine | acugcagugaaggcacuugu | 25 |
| miR-92 | bovine | uauugcacuugucccggccugu | 32 |
| miR-92b | human | uauugcacucgucccggccucc | 30 |
| | bovine | uauugcacucgucccggccucc | 36 |
| miR-146b-3p | human | ugagaacugaauuccauaggcu | 55 |
| miR-150 | human | ucucccaacccuuguaccagug | 62 |
| | bovine | ucucccaacccuuguaccagugu | 64 |
| miR-17-5p | human | caaagugcuuacagugcagguag | 22 |
| | bovine | caaagugcuuacagugcagguagu | 24 |
| | rat | caaagugcuuacagugcagguag | 65 |
| miR-92a | human | uauugcacuugucccggccugu | 28 |
| | bovine | uauugcacuugucccggccugu | 34 |
| | rat | uauugcacuugucccggccug | 66 |
| miR-146a | human | ugagaacugaauuccauggguu | 53 |
| | bovine | ugagaacugaauuccauagguugu | 58 |
| | rat | ugagaacugaauuccauggguu | 67 |
| miR-16 | human | uagcagcacguaaauauuggcg | 68 |
| | rat | uagcagcacguaaauauuggcg | 69 |
| miR-16a | bovine | uagcagcacguaaauauuggug | 70 |
| miR-18a | human | uaaggugcaucuagugcagauag | 71 |
| | bovine | uaaggugcaucuagugcagaua | 72 |
| | rat | uaaggugcaucuagugcagauag | 73 |
| miR-19b | human | ugugcaaauccaugcaaaacuga | 74 |
| | bovine | ugugcaaauccaugcaaaacuga | 75 |
| | rat | ugugcaaauccaugcaaaacuga | 76 |
| miR-20a | human | uaaagugcuuauagugcagguag | 77 |
| | bovine | uaaagugcuuauagugcagguag | 78 |
| | rat | uaaagugcuuauagugcagguag | 79 |
| miR-21 | human | uagcuuaucagacugauguuga | 80 |
| | bovine | uagcuuaucagacugauguugacu | 81 |
| | rat | uagcuuaucagacugauguuga | 82 |
| miR-23a | human | aucacauugccagggauuucc | 83 |
| | bovine | aucacauugccagggauuucca | 84 |
| | rat | aucacauugccagggauuucc | 85 |
| miR-27a | human | uucacaguggcuaaguuccgc | 86 |
| | rat | uucacaguggcuaaguuccgc | 87 |
| miR-27a-3p | bovine | uucacaguggcuaaguuccg | 88 |
| miR-27a-5p | bovine | agggcuuagcugcuugugagca | 89 |
| miR-27b | human | uucacaguggcuaaguucugc | 90 |
| | bovine | uucacaguggcuaaguucugc | 91 |
| | rat | uucacaguggcuaaguucugc | 92 |
| miR-29a | human | uagcaccaucugaaaucgguua | 93 |
| | bovine | cuagcaccaucugaaaucgguua | 94 |
| | rat | uagcaccaucugaaaucgguua | 95 |

TABLE 2

| | Human or | | SEQ |
|---|---|---|---|
| miRNA | animal | Sequence | ID NO |
| miR-29b | human | uagcaccauuugaaaucaguguu | 96 |
| | bovine | uagcaccauuugaaaucaguguu | 97 |
| | rat | uagcaccauuugaaaucaguguu | 98 |
| miR-29c | human | uagcaccauuugaaaucgguua | 99 |
| | bovine | uagcaccauuugaaaucgguua | 100 |
| | rat | uagcaccauuugaaaucgguua | 101 |
| miR-29c* | human | ugaccgauuucuccugguguuc | 102 |
| | rat | ugaccgauuucuccugguguuc | 103 |
| miR-30a | human | uguaaacauccucgacuggaag | 104 |
| | bovine | uguaaacauccucgacuggaagcu | 105 |
| | rat | uguaaacauccucgacuggaag | 106 |
| miR-30c | human | uguaaacauccuacacucucagc | 107 |
| | bovine | uguaaacauccuacacucucagc | 108 |
| | rat | uguaaacauccuacacucucagc | 109 |
| miR-30d | human | uguaaacauccccgacuggaag | 110 |
| | bovine | uguaaacauccccgacuggaagcu | 111 |
| | rat | uguaaacauccccgacuggaag | 112 |
| miR-30e* | human | cuuucagucggauguuuacagc | 113 |
| | rat | cuuucagucggauguuuacagc | 114 |
| miR-33a | human | gugcauuguaguugcauugca | 115 |
| | bovine | gugcauuguaguugcauugca | 116 |
| miR-33 | rat | gugcauuguaguugcauugca | 117 |
| miR-34b | human | caaucacuaacuccacugccau | 118 |
| | bovine | aggcaguguaauuagcugauug | 119 |
| | rat | uaggcaguguaauuagcugauug | 120 |
| miR-93 | human | caaagugcuguucgugcagguag | 121 |
| | bovine | caaagugcuguucgugcaggua | 122 |
| | rat | caaagugcuguucgugcagguag | 123 |
| miR-100 | human | aacccguagauccgaacuugug | 124 |
| | bovine | aacccguagauccgaacuugg | 125 |
| | rat | aacccguagauccgaacuugug | 126 |
| miR-101 | human | uacaguacugugauaacugaa | 127 |
| miR-101a | bovine | uacaguacugugauaacugaa | 128 |
| | rat | uacaguacugugauaacugaa | 129 |
| miR-101b | rat | uacaguacugugauagcugaa | 130 |
| miR-106b | bovine | uaaagugcugacagugcagau | 131 |
| | rat | uaaagugcugacagugcagau | 132 |
| miR-130b | human | cagugcaaugaugaaagggcau | 133 |
| | bovine | cagugcaaugaugaaagggcau | 134 |
| | rat | cagugcaaugaugaaagggcau | 135 |
| miR-140-3p | human | uaccacaggguagaaccacgg | 136 |
| miR-140* | rat | uaccacaggguagaaccacgg | 137 |
| miR-141 | human | uaacacugucugguaaagaugg | 138 |
| | bovine | uaacacugucugguaaagaugg | 139 |
| | rat | uaacacugucugguaaagaugg | 140 |
| miR-143 | human | ugagaugaagcacuguagcuc | 141 |
| | bovine | ugagaugaagcacuguagcucg | 142 |
| | rat | ugagaugaagcacuguagcuca | 143 |

TABLE 3

| | Human or | | SEQ |
|---|---|---|---|
| miRNA | animal | Sequence | ID NO |
| miR-185 | human | uggagagaaaggcaguuccuga | 144 |
| | bovine | uggagagaaaggcaguuccuga | 145 |
| | rat | uggagagaaaggcaguuccuga | 146 |
| miR-186 | human | caaagaauucuccuuuugggcu | 147 |
| | bovine | caaagaauucuccuuuugggcu | 148 |
| | rat | caaagaauucuccuuuugggcu | 149 |
| miR-192 | human | cugaccuaugaauugacagcc | 150 |
| | bovine | cugaccuaugaauugacagccag | 151 |
| | rat | cugaccuaugaauugacagcc | 152 |
| miR-193a-3p | human | aacuggccuacaaagucccagu | 153 |
| | bovine | aacuggccuacaaagucccagu | 154 |
| miR-193 | rat | aacuggccuacaaagucccagu | 155 |
| miR-195 | human | uagcagcacagaaauauuggc | 156 |
| | bovine | uagcagcacagaaauauuggca | 157 |
| | rat | uagcagcacagaaauauuggc | 158 |
| miR-200a | human | uaacacugucugguaacgaugu | 159 |
| | bovine | uaacacugucugguaacgauguu | 160 |
| | rat | uaacacugucugguaacgaugu | 161 |
| miR-205 | human | uccuucauuccaccggagucug | 162 |
| | bovine | uccuucauuccaccggagucug | 163 |
| | rat | uccuucauuccaccggagucug | 164 |
| miR-208 | human | uugugcuugaucuaaccaugu | 165 |
| | rat | uugugcuugaucuaaccaugu | 166 |
| miR-219-5p | human | ugauuguccaaacgcaauucu | 167 |
| | rat | ugauuguccaaacgcaauucu | 168 |
| miR-221 | human | agcuacauugucugcuggguuuc | 169 |
| | bovine | agcuacauugucugcuggguuu | 170 |
| | rat | agcuacauugucugcuggguuuc | 171 |
| miR-301a | human | cagugcaauaguauugucaaagc | 172 |
| | bovine | cagugcaauaguauugucaaagcau | 173 |
| | rat | cagugcaauaguauugucaaagc | 174 |
| miR-322 | rat | cagcagcaauucauguuuugga | 175 |
| miR-340 | human | uuauaaagcaaugagacugauu | 176 |
| | bovine | uccgucucaguuacuuuauagcc | 177 |
| miR-340-5p | rat | uuauaaagcaaugagacugauu | 178 |
| miR-361 | human | uuaucagaaucuccagggguac | 179 |
| | bovine | uuaucagaaucuccagggguac | 180 |
| | rat | uuaucagaaucuccagggguac | 181 |
| miR-429 | human | uaauacugucugguaaaaccgu | 182 |
| | bovine | uaauacugucugguaaugccgu | 183 |
| | rat | uaauacugucugguaaugccgu | 184 |
| miR-455 | human | uaugugccuuuggacuacaucg | 185 |
| | bovine | uaugugccuuuggacuacauc | 186 |
| | rat | uaugugccuuuggacuacaucg | 187 |
| miR-466b | rat | uauguguguguguauguccaug | 188 |
| miR-497 | human | cagcagcacacugugguuugu | 189 |
| | bovine | cagcagcacacugugguuugua | 190 |
| | rat | cagcagcacacugugguuugua | 191 |

TABLE 4

| | Human or | | SEQ |
|---|---|---|---|
| miRNA | animal | Sequence | ID NO |
| miR-500 | human | uaauccuugcuaccugggugaga | 192 |
| | bovine | uaauccuugcuaccugggugaga | 193 |
| | rat | aaugcaccugggcaaggguuca | 194 |
| miR-503 | human | uagcagcgggaacaguucugcag | 195 |
| | rat | uagcagcgggaacaguacugcag | 196 |
| miR-532 | bovine | caugccuugaguguaggaccgu | 198 |
| miR-532-5p | human | caugccuugaguguaggaccgu | 197 |
| | rat | caugccuugaguguaggacugu | 199 |
| miR-542-3p | human | ugugacagauugauaacugaaa | 200 |
| | rat | ugugacagauugauaacugaaa | 201 |
| let-7a | human | ugagguaguagguuguauaguu | 202 |
| | bovine | ugagguaguagguuguauaguu | 203 |
| | rat | ugagguaguagguuguauaguu | 204 |
| let-7a* | human | cuauacaaucuacugucuuuc | 205 |
| | bovine | cuauacaaucuacugucuuuc | 206 |
| | rat | ugagguaguagguuguauaguu | 207 |
| let-7b | human | ugagguaguagguugugugguu | 208 |
| | bovine | ugagguaguagguugugugguu | 209 |
| | rat | ugagguaguagguugugugguu | 210 |
| let-7c | human | ugagguaguagguuguaugguu | 211 |
| | bovine | ugagguaguagguuguaugguu | 212 |
| | rat | ugagguaguagguuguaugguu | 213 |
| let-7d | human | agagguaguagguugcauaguu | 214 |
| | bovine | agagguaguagguugcauaguu | 215 |
| | rat | agagguaguagguugcauaguu | 216 |
| let-7e | human | ugagguaggagguuguauaguu | 217 |
| | bovine | ugagguaggagguuguauagu | 218 |
| | rat | ugagguaggagguuguauaguu | 219 |
| let-7f | human | ugagguaguagauuguauaguu | 220 |
| | bovine | ugagguaguagauuguauaguu | 221 |
| | rat | ugagguaguagauuguauaguu | 222 |
| let-7i | human | ugagguaguaguuugugcuguu | 38 |
| | bovine | ugagguaguaguuugugcuguu | 40 |
| | rat | ugagguaguaguuugugcuguu | 223 |
| miR-10a | human | uacccuguagauccgaauuugug | 224 |
| | bovine | uacccuguagauccgaauuugug | 225 |
| miR-10a-5p | rat | uacccuguagauccgaauuugug | 226 |
| miR-10b | human | uacccuguagaaccgaauuugug | 227 |
| | bovine | uacccuguagaaccgaauuugug | 228 |
| | rat | cccuguagaaccgaauuugugu | 229 |
| miR-15b | human | uagcagcacaucaugguuuaca | 230 |
| | bovine | uagcagcacaucaugguuuaca | 231 |
| | rat | uagcagcacaucaugguuuaca | 232 |
| miR-19a | human | ugugcaaaucuaugcaaaacuga | 233 |
| | bovine | ugugcaaaucuaugcaaaacuga | 234 |
| | rat | ugugcaaaucuaugcaaaacuga | 235 |
| miR-20a* | human | acugcauuaugagcacuuaaag | 236 |
| | rat | acugcauuacgagcacuuaca | 237 |
| miR-22 | human | aagcugccaguugaagaacugu | 238 |

TABLE 5

| | Human or | | SEQ |
|---|---|---|---|
| miRNA | animal | Sequence | ID NO |
| miR-22-3p | bovine | aagcugccaguugaagaacug | 239 |
| miR-22 | rat | aagcugccaguugaagaacugu | 240 |
| miR-23b | human | aucacauugccagggauuacc | 241 |
| | rat | aucacauugccagggauuacc | 242 |
| miR-23b-5p | bovine | ggguuccuggcaugcugauuu | 243 |
| miR-23b-3p | bovine | aucacauugccagggauuaccac | 244 |
| miR-24 | human | uggcucaguucagcaggaacag | 245 |
| | bovine | gugccuacugagcugauaucagu | 246 |
| | rat | uggcucaguucagcaggaacag | 247 |
| miR-25 | human | cauugcacuugucucggucuga | 248 |
| | bovine | cauugcacuugucucggucuga | 249 |
| | rat | cauugcacuugucucggucuga | 250 |
| miR-26a | human | uucaaguaauccaggauaggcu | 251 |
| | bovine | uucaaguaauccaggauaggcu | 252 |
| | rat | uucaaguaauccaggauaggcu | 253 |
| miR-26b | human | uucaaguaauucaggauaggu | 254 |
| | bovine | uucaaguaauucaggauagguu | 472 |
| | rat | uucaaguaauucaggauaggu | 255 |
| miR-28 | human | aaggagcucacagucuauugag | 256 |
| | bovine | aaggagcucacagucuauugag | 257 |
| | rat | aaggagcucacagucuauugag | 258 |
| miR-30a* | human | cuuucagucggauguuugcagc | 259 |
| | rat | cuuucagucggauguuugcagc | 260 |
| miR-30b | human | uguaaacauccuacacucagcu | 261 |
| miR-30b-5p | bovine | uguaaacauccuacacucagcu | 262 |
| | rat | uguaaacauccuacacucagcu | 263 |
| miR-30c-1* | human | cugggagaggguuguuuacucc | 264 |
| | rat | cugggagaggguuguuuacucc | 265 |
| miR-30c-2* | human | cugggagaaggcuguuuacucu | 266 |
| | rat | cugggagaaggcuguuuacucu | 267 |
| miR-30e | human | uguaaacauccuugacuggaag | 268 |
| | rat | uguaaacauccuugacuggaag | 270 |
| miR-30e-5p | bovine | uguaaacauccuugacuggaagcu | 269 |
| miR-31 | human | aggcaagaugcuggcauagcu | 271 |
| | bovine | aggcaagaugcuggcauagcu | 272 |
| | rat | aggcaagaugcuggcauagcug | 273 |
| miR-34a | human | uggcagugucuuagcugguugu | 274 |
| | bovine | uggcagugucuuagcugguugu | 275 |
| | rat | uggcagugucuuagcugguugu | 276 |
| miR-96 | human | uuuggcacuagcacauuuuugcu | 277 |
| | bovine | uuuggcacuagcacauuuuugcu | 278 |
| | rat | uuuggcacuagcacauuuuugcu | 279 |
| miR-98 | human | ugagguaguaaguuguauuguu | 280 |
| | bovine | ugagguaguaaguuguauuguu | 281 |
| | rat | ugagguaguaaguuguauuguu | 282 |
| miR-99a | human | aacccguagauccgaucuugug | 283 |
| | bovine | aacccguagauccgaucuugu | 284 |
| | rat | aacccguagauccgaucuugug | 285 |

TABLE 6

| | Human or | | SEQ |
|---|---|---|---|
| miRNA | animal | Sequence | ID NO |
| miR-99b | human | cacccguagaaccgaccuugcg | 286 |
| | bovine | cacccguagaaccgaccuugcg | 287 |
| | rat | cacccguagaaccgaccuugcg | 288 |
| miR-103 | human | agcagcauuguacagggcuauga | 289 |
| | bovine | agcagcauuguacagggcuauga | 290 |
| | rat | agcagcauuguacagggcuauga | 291 |
| miR-107 | human | agcagcauuguacagggcuauca | 292 |
| | bovine | agcagcauuguacagggcuauc | 293 |
| | rat | agcagcauuguacagggcuauca | 294 |
| miR-125a-3p | human | acaggugagguucuugggagcc | 43 |
| | rat | acaggugagguucuugggagcc | 295 |
| miR-125a-5p | human | ucccugagacccuuuaaccuguga | 42 |
| | rat | ucccugagacccuuuaaccuguga | 296 |
| miR-125a | bovine | ucccugagacccuuuaaccugug | 48 |
| miR-125b | human | ucccugagacccuaacuuguga | 46 |
| | bovine | ucccugagacccuaacuuguga | 51 |
| miR-125b-5p | rat | ucccugagacccuaacuuguga | 297 |
| miR-125b-1* | human | acggguuaggcucuugggagcu | 298 |
| miR-125b-3p | rat | acggguuaggcucuugggagcu | 299 |
| miR-128 | human | ucacagugaaccggucucuuu | 300 |
| | bovine | ucacagugaaccggucucuuu | 301 |
| | rat | ucacagugaaccggucucuuu | 302 |
| miR-130a | human | cagugcaauguuaaaagggcau | 303 |
| | bovine | cagugcaauguuaaaagggcau | 304 |
| | rat | cagugcaauguuaaaagggcau | 305 |
| miR-133a | human | uuugguccccuucaaccagcug | 306 |
| | bovine | uuugguccccuucaaccagcug | 307 |
| | rat | uuugguccccuucaaccagcug | 308 |
| miR-133b | human | uuugguccccuucaaccagcua | 309 |
| | bovine | uuugguccccuucaaccagcua | 310 |
| | rat | uuugguccccuucaaccagcua | 311 |
| miR-134 | human | ugugacugguugaccagagggg | 312 |
| | bovine | ugugacugguugaccagagugg | 313 |
| | rat | ugugacugguugaccagagggg | 314 |
| miR-139-3p | human | ggagacgcggcccuguuggagu | 315 |
| | rat | uggagacgcggcccuguuggag | 316 |
| miR-140 | human | cagugguuuuacccuaugguag | 317 |
| | bovine | uaccacaggguagaaccacgga | 318 |
| | rat | cagugguuuuacccuaugguag | 319 |
| miR-146b | human | ugagaacugaauuccauaggcu | 55 |
| | bovine | ugagaacugaauuccauaggcugu | 60 |
| | rat | ugagaacugaauuccauaggcugu | 320 |
| miR-148b | human | ucagugcaucacagaacuuugu | 321 |
| | bovine | ucagugcaucacagaacuuugu | 322 |
| miR-148b-3p | rat | ucagugcaucacagaacuuugu | 323 |
| miR-151 | human | ucgaggagcucacagucuagu | 324 |
| | bovine | cuagacugaagcuccuugagg | 325 |
| | rat | cuagacugaagcuccuugagg | 326 |

TABLE 7

| | Human or | | SEQ |
|---|---|---|---|
| miRNA | animal | Sequence | ID NO |
| miR-152 | human | ucagugcaugacagaacuugg | 327 |
| | bovine | ucagugcaugacagaacuuggg | 328 |
| | rat | ucagugcaugacagaacuugg | 329 |
| miR-181a | human | aacauucaacgcugucggugagu | 7 |
| | bovine | aacauucaacgcugucggugaguu | 13 |
| | rat | aacauucaacgcugucggugagu | 330 |
| miR-181a* | human | accaucgaccguugauuguacc | 331 |
| | rat | accaucgaccguugauuguacc | 332 |
| miR-181b | human | aacauucauugcugucggugggu | 10 |
| | bovine | aacauucauugcugucgguggguu | 16 |
| | rat | aacauucauugcugucggugggu | 333 |
| miR-181c | human | aacauucaaccugucggugagu | 334 |
| | bovine | aacauucaaccugucggugaguuu | 335 |
| | rat | aacauucaaccugucggugagu | 336 |
| miR-181d | human | aacauucauuguugucggugggu | 337 |
| | bovine | aacauucauuguugucggugggu | 338 |
| | rat | aacauucauuguugucggugggu | 339 |
| miR-182 | human | uuuggcaaugguagaacucacacu | 340 |
| | bovine | uuuggcaaugguagaacucacacu | 341 |
| | rat | uuuggcaaugguagaacucacaccg | 342 |
| miR-183 | human | uauggcacugguagaauucacu | 343 |
| | bovine | uauggcacugguagaauucacug | 344 |
| | rat | uauggcacugguagaauucacu | 345 |
| miR-188 | human | caucccuugcaugguggaggg | 346 |
| | bovine | caucccuugcaugguggagggu | 347 |
| | rat | caucccuugcaugguggaggg | 348 |
| miR-196c | rat | uagguaguuucguguuguuggg | 349 |
| miR-199a-3p | human | acaguagucugcacauugguua | 350 |
| | bovine | acaguagucugcacauugguua | 351 |
| | rat | acaguagucugcacauugguua | 352 |
| miR-200b | human | uaauacugccugguaaugauga | 353 |
| | bovine | uaauacugccugguaaugaug | 354 |
| | rat | uaauacugccugguaaugaugac | 355 |
| miR-200c | human | uaauacugccggguaaugaugga | 356 |
| | bovine | uaauacugccggguaaugaugga | 357 |
| | rat | uaauacugccggguaaugaugg | 358 |
| miR-203 | human | gugaaauguuuaggaccacuag | 359 |
| | rat | gugaaauguuuaggaccacuag | 360 |
| miR-204 | human | uucccuuugucauccuaugccu | 361 |
| | bovine | uucccuuugucauccuaugccu | 362 |
| | rat | uucccuuugucauccuaugccu | 363 |
| miR-206 | human | uggaauguaaggaagugugugg | 364 |
| | bovine | uggaauguaaggaagugugugg | 365 |
| | rat | uggaauguaaggaagugugugg | 366 |
| miR-210 | human | cugugcgugugacagcggcuga | 367 |
| | bovine | acugugcgugugacagcggcuga | 368 |
| | rat | cugugcgugugacagcggcuga | 369 |

TABLE 8

| | Human or | | SEQ |
|---|---|---|---|
| miRNA | animal | Sequence | ID NO |
| miR-212 | human | uaacagucuccagucacgguu | 370 |
| | bovine | accuuggcucuagacugcuuacu | 371 |
| | rat | uaacagucuccagucacggcca | 372 |
| miR-214 | human | acagcaggcacagacaggcagu | 373 |
| | bovine | acagcaaggcacagacaggcagu | 374 |
| | rat | acagcaggcacagacaggcag | 375 |
| miR-222 | human | agcuacaucuggcuacugggu | 376 |
| | bovine | agcuacaucuggcuacugggu | 377 |
| | rat | agcuacaucuggcuacugggu | 378 |
| miR-223 | human | ugucaguuugucaaauacccca | 18 |
| | bovine | ugucaguuugucaaauacccca | 20 |
| | rat | ugucaguuugucaaauacccc | 379 |
| miR-290 | rat | cucaaacuaugggggcacuuuuu | 380 |
| miR-291a-5p | rat | caucaaaguggaggcccucucu | 381 |
| miR-292-5p | rat | acucaaacugggggcucuuuug | 382 |
| miR-294 | rat | cucaaauggaggcccuaucu | 383 |
| miR-296-5p | human | agggcccccccucaauccgug | 384 |
| miR-296* | rat | agggcccccccucaauccugu | 385 |
| miR-320a | human | aaaagcuggguugagagggcga | 386 |
| miR-320 | bovine | aaaagcuggguugagagggcga | 387 |
| | rat | aaaagcuggguugagagggcga | 388 |
| miR-324-3p | human | acugccccaggugcugcugg | 389 |
| | rat | ccacugccccaggugcugcugg | 390 |
| miR-324 | bovine | cgcauccccuagggcauuggugu | 392 |
| miR-324-5p | human | cgcauccccuagggcauuggugu | 391 |
| | rat | cgcauccccuagggcauuggugu | 393 |
| miR-327 | rat | ccuugaggggcaugagggu | 394 |
| miR-328 | human | cuggccucucugcccuuccgu | 395 |
| | bovine | cuggcccucucugcccuuccgu | 396 |
| | rat | cuggcccucucugcccuuccgu | 397 |
| miR-331 | human | gccccugggccuauccuagaa | 398 |
| | bovine | gccccugggccuauccuagaa | 399 |
| | rat | gccccugggccuauccuagaa | 400 |
| miR-340-3p | rat | uccgucucaguuacuuuauagcc | 403 |
| miR-341 | rat | ucggucgaucggucggucggu | 404 |
| miR-342 | bovine | ucucacacagaaaucgcacccaucu | 406 |
| miR-342-3p | human | ucucacacagaaaucgcacccgu | 405 |
| | rat | ucucacacagaaaucgcacccgu | 407 |
| miR-345 | human | gcugacuccuaguccagggcuc | 408 |
| miR-345-5p | bovine | gcugacuccuaguccagugcu | 409 |
| | rat | ugcugaccccuaguccagugc | 410 |
| miR-347 | rat | ugucccucugggucgccca | 411 |
| miR-352 | rat | agaguaguagguugcauagua | 412 |
| miR-365 | human | uaaugccccuaaaaauccuuau | 413 |
| | rat | uaaugccccuaaaaauccuuau | 415 |
| miR-365-3p | bovine | uaaugccccuaaaaauccuuau | 414 |

TABLE 9

| | Human or | | SEQ |
|---|---|---|---|
| miRNA | animal | Sequence | ID NO |
| miR-370 | human | gccugcugggguggaaccuggu | 416 |
| | bovine | gccugcugggguggaaccuggu | 417 |
| | rat | gccugcugggguggaaccugguu | 418 |
| miR-375 | human | uuuguucguucggcucgcugua | 419 |
| | bovine | uuuuguucguucggcucgcguga | 420 |
| | rat | uuuguucguucggcucgcguga | 421 |
| miR-378 | human | acuggacuuggagucagaagg | 422 |
| | bovine | acuggacuuggagucagaaggc | 423 |
| | rat | acuggacuuggagucagaagg | 424 |
| miR-378* | human | cuccugacuccagguccugugu | 425 |
| | rat | cuccugacuccagguccugugu | 426 |
| miR-425 | human | aaugacacgaucacucccguuga | 427 |
| | bovine | augacacgaucacucccguuga | 428 |
| | rat | aaugacacgaucacucccguuga | 429 |
| miR-465 | rat | uauuuagaacggugcuggugu | 430 |
| miR-483 | human | ucacuccucuccucccgucuu | 431 |
| | bovine | ucacuccucuccucccgucuu | 432 |
| | rat | ucacuccuccccucccgucuugu | 433 |
| miR-484 | human | ucaggcucaguccccucccgau | 434 |
| | bovine | ucaggcucaguccccucccgau | 435 |
| | rat | ucaggcucaguccccucccgau | 436 |
| miR-494 | human | ugaaacauacacgggaaaccuc | 437 |
| | bovine | ugaaacauacacgggaaaccuc | 438 |
| | rat | ugaaacauacacgggaaaccu | 439 |
| miR-542-5p | human | ucggggaucaucaugucacgaga | 440 |
| | bovine | ucggggaucaucaugucacgag | 441 |
| | rat | cucggggaucaucaugucacga | 442 |
| miR-652 | human | aauggcgccacuaggguugug | 443 |
| | rat | aauggcgccacuaggguugug | 444 |
| miR-672 | human | ugagguugguguacuguguguga | 445 |
| | rat | ugagguugguguacuguguguga | 446 |
| miR-685 | bovine | ucaauggcugaggugagguac | 447 |
| | rat | ucaauggcugaggugaggcac | 448 |
| miR-760 | human | cggcucugggucugugggga | 449 |
| | bovine | ccccucaguccaccagagcccg | 450 |
| miR-760-3p | rat | cggcucugggucugugggga | 451 |
| miR-872 | human | aagguuacuuguuaguucagg | 452 |
| | rat | aagguuacuuguuaguucagg | 453 |
| miR-874 | human | cugcccuggcccgagggaccga | 454 |
| | bovine | cugcccuggcccgagggaccga | 455 |
| | rat | cugcccuggcccgagggaccga | 456 |
| miR-1224-5p | human | gugaggacucgggaggugg | 457 |
| miR-1224 | bovine | gugaggacucgggagguggag | 458 |
| | rat | gugaggacuggggagguggag | 459 |
| miR-193* | rat | ugggucuuugcgggcaagauga | 460 |
| miR-193a-5p | human | ugggucuuugcgggcgagauga | 461 |
| | bovine | ugggucuuugcgggcgagauga | 462 |
| miR-409-3p | human | gaauguugcucggugaaccccu | 463 |
| | rat | aauguugcucggugaacccc | 464 |

TABLE 10

| | Human or | | SEQ |
|---|---|---|---|
| miRNA | animal | Sequence | ID NO |
| miR-409 | bovine | agguuacccgagcaacuuugcau | 465 |
| miR-664 | human | uaucauuuauccccagccuaca | 466 |
| | bovine | caggcugggguguguguggaug | 467 |
| | rat | uauucauuuacuccccagccua | 468 |
| miR-877 | human | guagaggagauggcgcaggg | 469 |
| | bovine | guagaggagauggcgcaggg | 470 |
| | rat | guagaggagauggcgcaggg | 471 |
| miR-15a | human | uagcagcacauaaugguuugug | 473 |
| | bovine | uagcagcacauaaugguuugu | 474 |
| miR-16b | bovine | uagcagcacguaaauauuggc | 475 |
| miR-30f | bovine | uguaaacacccuacacucucagcu | 476 |
| miR-106 | bovine | aaaagugcuuacagugcaggua | 477 |
| miR-126 | human | ucguaccgugaguaauaaugcg | 478 |
| | bovine | cguaccgugaguaauaaugcg | 479 |
| | rat | ucguaccgugaguaauaaugcg | 480 |
| miR-129-3p | human | aagcccuuaccccaaaaagcau | 481 |
| | bovine | aagcccuuaccccaaaaagcau | 482 |
| miR-184 | human | uggacggagaacugauaagggu | 483 |
| | bovine | uggacggagaacugauaagggu | 484 |
| | rat | uggacggagaacugauaagggu | 485 |
| miR-196a | human | uagguaguuucauguuguuggg | 486 |
| | bovine | uagguaguuucauguuguuggg | 487 |
| | rat | uagguaguuucauguuguuggg | 488 |
| miR-338 | human | uccagcaucagugauuuuguug | 489 |
| | bovine | uccagcaucagugauuuuguuga | 490 |
| | rat | uccagcaucagugauuuuguuga | 491 |
| miR-362-5p | human | aauccuuggaaccuaggugugagu | 492 |
| | bovine | aauccuuggaaccuaggugugagu | 493 |
| miR-362 | rat | aauccuuggaaccuaggugugaau | 494 |
| miR-452 | human | aacuguuugcagaggaaacuga | 495 |
| | bovine | uguuugcagaggaaacugagac | 496 |
| miR-486 | human | uccuguacugagcugccccgag | 497 |
| | bovine | uccuguacugagcugccccgag | 498 |
| miR-584 | human | uuaugguuugccugggacugag | 499 |
| | bovine | ugguuugccugggacugag | 500 |
| miR-708 | human | aaggagcuuacaaucuagcuggg | 501 |
| | bovine | aaggagcuuacaaucuagcuggg | 502 |
| | rat | aaggagcuuacaaucuagcuggg | 503 |
| miR-1300b | bovine | ucgagaaggaggcugcug | 504 |
| miR-1307 | human | acucggcguggcgucggucgug | 401 |
| | bovine | acucggcguggcgucggucgug | 402 |

The miRNA is not limited to those having the aforementioned sequences, the miRNA may include substitutions, deletions, insertions, additions or inversions of one or several nucleotides, so long as the miRNA has the function as the miRNA, i.e., the miRNA can regulate expression of target genes. Specifically, examples of such a miRNA include RNAs having a nucleotide sequence showing a homology of 80% or more, preferably 90% or more, more preferably 95% or more, to any of the aforementioned sequences.

The amount of miRNA may be an absolute amount or a relative amount. The relative amount may be a relative amount based on an average amount in animals, or may be a relative amount observed after ingestion of a diet based on the amount observed before the ingestion. For the measurement of the amount of nucleic acid, methods usually used for measurement of miRNA amount such as quantitative reverse transcription PCR (qRT-PCR) can be employed. The amount of miRNA can also be measured by the microarray method. As for extraction of miRNA from milk, methods usually used for extraction of miRNA can be employed, and a commercially available miRNA isolation kit can also be used.

Further, amount of miRNA present in milk can also be indirectly measured by measuring expression amount of the miRNA in mammary glandular cells.

Correlation of miRNA profiles in milk of a mammal and a diet ingested by the mammal or a substance contained in the diet is examined. The correlation of the miRNA profiles in milk of a mammal and a diet ingested by the mammal or a substance contained in the diet refers to correlation of the miRNA profile and presence or absence of the substance or amount of the substance. For example, if amounts of one or more kinds of miRNAs in milk of an animal which has ingested a certain substance are larger or smaller than those observed in the animal which has not ingested the substance, the substance and the miRNA profiles have positive or negative correlation, respectively. Further, if ingestion of a certain substance does not affect miRNA profiles, the substance and the miRNA profiles do not correlate with each other.

Specifically, for example, when miRNA profiles in milk observed before and after ingestion of a diet are compared, amount or amounts of one kind, preferably two kinds or more, more preferably five kinds or more, of miRNAs observed after the ingestion are larger than those observed before the ingestion, it is judged that the diet increases amounts of miRNAs existing in milk.

Further, when miRNA profiles in milk observed before and after ingestion of a diet are compared, amount or amounts of one kind, preferably two kinds or more, more preferably five kinds or more, of miRNAs observed after the ingestion are smaller than those observed before the ingestion, it is judged that the diet decreases amounts of miRNAs existing in milk.

Furthermore, measurement of miRNA profiles before ingestion of a diet is not indispensable, and correlation of a diet and amount of miRNA can also be examined by comparing a miRNA profile measured after ingestion of a diet with ordinary miRNA profiles of an objective mammal measured beforehand.

In another embodiment, miRNA profiles in milk and miRNA profiles in serum or plasma are compared, and if amount of miRNA contained in both of milk and serum or plasma is increased by ingestion of the diet at a higher degree in milk as compared to that observed in serum or plasma, it is judged that the diet increases amount of the miRNA present in milk. The degree of increase in amount of miRNA in milk is, for example, 1.2 times or more, preferably 2 times or more, more preferably 5 times or more, still more preferably 10 times or more, of that observed in serum or plasma.

Further, when miRNA profiles in milk and miRNA profiles in serum or plasma are compared, if amount of a miRNA contained in both of milk and serum or plasma is decreased by ingestion of the diet at a lower degree in milk as compared to that observed in serum or plasma, it is judged that the diet decreases amount of the miRNA present in milk. The degree of decrease in amount of miRNA in milk is, for example, 0.8 times or less, preferably 0.5 times or less, more preferably 0.2 times or less, still more preferably 0.1 times or less, of that observed in serum or plasma.

The diet may consist of a single substance or may be a composition, so long as it can be orally ingested. Further, “before ingestion” and “after ingestion” may mean “before and after one time of ingestion of diet”, or “before and after two or more times of ingestion of diet”. Further, two or more times of ingestion of diet may be two or more times of ingestion of the same diet, or ingestion of two or more kinds of diets.

The diet may be ingested according to a planned scheme or freely ingested. In the latter case, correlation of the diet and miRNA profiles in milk can be examined by hearing content of ingested diet in the case of human. When the diet is ingested or administered according to a planned scheme, the diet can be considered as a “test sample”. The diet may be a usual diet or a usual diet containing a test substance. Amount of diet to be ingested, time of ingestion, and number of times of ingestion are not particularly limited.

If a diet that increases amount of miRNA in milk is chosen, a substance that is contained in the diet and increases amount of the miRNA in milk can be identified in the same manner as that mentioned above. Further, if a diet that decreases amount of miRNA in milk is chosen, a substance that is contained in the diet and decreases amount of the miRNA in milk can be identified in the same manner as that mentioned above.

If a diet or a substance that increases or decreases amount of miRNA in milk is identified, a diet that increases or decreases amount of the miRNA in milk can be designed. That is, it is thought that a diet that increases amount of miRNA in milk or a substance contained therein is preferred for production of milk having an immunostimulating action, and a diet that decreases amount of miRNA in milk or a substance contained therein is not preferred for production of milk having an immunostimulating action.

Further, it is thought that a diet that decreases amount of miRNA in milk or a substance contained therein is preferred for production of milk having an immunosuppressive action, and a diet that increases amount of miRNA in milk or a substance contained therein is not preferred for production of milk having an immunosuppressive action.

Screening for a diet or a substance providing production of breast milk having an immunoregulatory action, or a diet or a substance unsuitable for production of breast milk having an immunoregulatory action can be performed as described above. As shown in the examples described later, presence of various kinds of miRNAs was confirmed in colostrum of rat and bovine. This supports the concept of the present invention that it is expected that oral administration of miRNA provides an immunoregulatory action. Further, as shown in the examples described later, when Bifidobacterium bacteria (Bifidobacterium longum) were orally administered to rats, amounts of 52 kinds of miRNAs increased.

It is known that Bifidobacterium bacteria function as probiotics, and have, in particular, an immunoregulatory action. Therefore, the fact that the administration of the Bifidobacterium bacteria increased amounts of miRNAs in milk also supports the involvement of miRNAs in milk in immunoregulation. Demonstration of increase in amounts of miRNAs in milk induced by administration of the Bifidobacterium bacteria, i.e., correlation of the Bifidobacterium bacteria and miRNA profiles, shows that the screening method of the present invention is feasible. Further, although there were also miRNAs of which amounts in milk were not changed by administration of the Bifidobacterium bacteria, a possibility that amounts of those miRNAs may be increased by another kind of diet or a substance contained therein is not denied.

As probiotic functions of Bifidobacterium bacteria, there are known prophylaxis or amelioration of respiratory tract infection, acute infectious diarrhea, antibiotic-associated diarrhea, Clostridium dificile-associated diarrhea, necrotising enterocolitis, traveler's diarrhea, Helicobacter pylori infection, and so forth (The Journal of Nutrition, 2010 March; 140(3):698S-712S. Epub 2010 Jan. 27). It is suggested that miRNA of which amount in milk is increased by administration of Bifidobacterium bacteria not only regulates immunity, but also exhibits functions similar to the aforementioned probiotic functions in animals that ingested them.

By giving a diet or a substance that increases amount of miRNA in milk chosen as described above to a mammal, and collecting milk from the animal, milk having an immunostimulating action or milk of which immunostimulating action is enhanced can be obtained. Further, by reducing or avoiding ingestion by a mammal of a diet or a substance that decreases amount of miRNA in milk chosen as described above, an immunostimulating action of milk can be enhanced, or decrease of an immunostimulating action can be prevented.

Further, ingestion of a diet or a substance that increases amount of miRNA in milk and reduction or avoidance of ingestion of a diet or a substance that decreases amount of miRNA in milk may be combined. Further, by giving a diet or a substance that decreases amount of miRNA in milk chosen as described above to a mammal, and collecting milk from the animal, milk having an immunosuppressive action or milk of which immunostimulating action is decreased can be obtained. Further, by reducing or avoiding ingestion by a mammal of a diet or a substance that increases amount of miRNA in milk chosen as described above, an immunosuppressive action of milk can be enhanced, or an immunostimulating action of milk can be decreased. Further, ingestion of a diet or a substance that decreases amount of miRNA in milk and reduction or avoidance of ingestion of a diet or a substance that increases amount of miRNA in milk may be combined.

By processing milk having an immunoregulatory action obtained as described above, dairy products having an immunoregulatory action can be produced.

Type of the dairy products is not particularly limited, so long as miRNAs can exist in it with maintaining the functions thereof, and examples include processed milk, infant formula, milk beverages, powdered infant formula, fermented milk, cream, butter, cheese, ice cream, and so forth. As the dairy product, a dairy product for infants or little children is preferred.

According to the present invention, there was demonstrated presence in milk of miRNAs, especially miRNAs which have been known to participate in enhancement of immunity, such as development of immunity, antiallergy, anti-inflammation, and defense against infection. In addition, it is well known that breast milk gives an immunostimulating action to an infant who ingested it. Therefore, it is rationally predicted that the miRNA participating in immunoregulation can regulate immunity of organism such as human who ingested it. Since miRNA is a substance that regulates expression of various genes, it is considered that transfer of such regulatory molecules from a mother to an infant is extremely significant for, in particular, infants having an underdeveloped immune system.

Another aspect of the present invention is a composition for oral ingestion having an immunostimulating action, which is prepared by adding miRNA to a base for composition for oral ingestion.

Examples of the miRNA include miR-10, miR-15, miR-16, miR-17, miR-18, miR-19, miR-20, miR-21, miR-22, miR-23, miR-24, miR-25, miR-26, miR-27, miR-28, miR-29, miR-30, miR-31, miR-33, miR-34, miR-92, miR-93, miR-96, miR-98, miR-99, miR-100, miR-101, miR-103, miR-106, miR-107, miR-125, miR-126, miR-128, miR-129, miR-130, miR-133, miR-134, miR-139, miR-140, miR-141, miR-143, miR-146, miR-148, miR-151, miR-152, miR-155, miR-181, miR-182, miR-183, miR-184, miR-185, miR-186, miR-188, miR-192, miR-193, miR-195, miR-196, miR-199, miR-200, miR-203, miR-204, miR-205, miR-206, miR-210, miR-212, miR-214, miR-218, miR-219, miR-221, miR-222, miR-223, miR-290, miR-291, miR-292, miR-294, miR-296, miR-301, miR-320, miR-322, miR-324, miR-327, miR-328, miR-331, miR-338, miR-340, miR-341, miR-342, miR-345, miR-347, miR-352, miR-361, miR-362, miR-365, miR-370, miR-375, miR-378, miR-409, miR-425, miR-429, miR-452, miR-455, miR-465, miR-466, miR-483, miR-484, miR-486, miR-494, miR-497, miR-500, miR-503, miR-532, miR-542, miR-584, miR-652, miR-664, miR-672, miR-685, miR-708, miR-760, miR-872, miR-874, miR-877, miR-1224, miR-1300, miR-1307, let-7a, let-7b, let-7c, let-7d, le-7e, let-7f, let-7i, and so forth.

Among the aforementioned miRNAs, miR-15, miR-16, miR-17, miR-18, miR-19, miR-20, miR-21, miR-23, miR-24, miR-26, miR-27, miR-29, miR-30, miR-33, miR-34, miR-92, miR-93, miR-99, miR-100, miR-101, miR-106, miR-107, miR-125, miR-130, miR-140, miR-141, miR-143, miR-146, miR-155, miR-181, miR-185, miR-186, miR-192, miR-193, miR-195, miR-200, miR-205, miR-210, miR-218, miR-219, miR-221, miR-222, miR-223, miR-301, miR-322, miR-340, miR-361, miR-370, miR-429, miR-455, miR-466, miR-497, miR-500, miR-503, miR-532, miR-542, let-7d, and let-7i are preferred, and miR-15, miR-16, miR-19, miR-21, miR-23, miR-24, miR-26, miR-27, miR-30, miR-34, miR-99, miR-106, miR-107, miR-125, miR-130, miR-140, miR-181, miR-193, miR-210, miR-222, miR-223, miR-361, miR-370, miR-429, miR-500, miR-532, let-7d, and let-7i are more preferred.

The miRNA may consist of a single kind of miRNA or arbitrary two or more kinds of miRNAs.

The base for composition for oral ingestion is not particularly limited so long as an orally ingestible or administrable base in which miRNA can exist with maintaining functions thereof is chosen, and examples include foodstuffs, drinks, drug bases, animal feeds, and so forth.

Foodstuffs may be in any form, and include drinks. Foodstuffs include foodstuffs for adults, foodstuffs for infants, foodstuffs for little children, and so forth.

Examples of the foodstuffs for adults include enteral nutrients, fluid diets such as concentrated fluid diets, nutritional supplementary foods, and so forth.

Examples of the foodstuffs for infants or the foodstuffs for little children include, for example, modified milks (for example, infant formula, infant formula for low birth weight infants, follow-up formula, etc. as well as infant formula for allergic infants, non-lactose milk, special milk for inborn errors of metabolism infants, etc., and dried milk prepared from these), powders for supplement of breast milk or powdered infant formula, baby food, and so forth.

The infant formula referred to here are foodstuffs produced by using milk or dairy products as main raw materials, and adding nutrients required for infants, and are mainly used as alternative food for breast milk in infancy, and as alternative food for breast milk or nutritional complementary food in childhood. Other examples thereof include foodstuffs produced for the purpose of contributing to nutritional ingestion suitable for infants with a specific inherent or acquired disease.

miRNA is relatively resistant to freeze-thaw, low pH such as acidic conditions of pH 1, and RNases such as RNase A and RNase T, and thus is suitable as an active ingredient to be added to foodstuffs. The stability at a low pH suggests that miRNA molecules are resistant to the infant's intragastric environment, and can be absorbed by the intestinal tract, which is one of the main immune organs of infants, and thus they can affect the immune system of infants. Further, storage and freeze-thaw of breast milk do not denature miRNA, and this is nutritionally important for low birth weight infants and hospitalized infants, who are usually given cryopreserved breast milk. Furthermore, the resistance of miRNA to RNases suggests that miRNA may exist in a complex such as exosome and microvesicle in breast milk.

From the aforementioned findings, it sounds that mothers give to infants such custom-made breast milk that the infants can adapt to the environment. There is a report suggesting that breast milk-derived exosomes increase the number of Foxp3+ CD4+ CD25+ regulatory T cells. If immunity-related miRNAs are contained in breast milk exosomes, they may possibly contribute to the increase in Foxp3+ CD4+ CD25+ regulatory T cells in the alimentary canal of infants. This is because the immunity-related miRNAs detected in breast milk such as miR-181a and miR-181b are highly expressed, and they are involved in T cell differentiation. Furthermore, since it is known that miR-181 and miR-155 abundantly contained in breast milk induce B cell differentiation, and there is almost no miR-150, which suppresses B cell differentiation, in breast milk, miRNAs in breast milk may induce differentiation of B cells.

Although content of miRNA in the composition is not particularly limited, and may be appropriately chosen, it is, for example, 10 to 10,000 ng/ml, preferably 20 to 10,000 ng/ml, more preferably 50 to 10,000 ng/ml, in total. Further, amount of miRNA to be ingested is, for example, 5 μg to 120 mg/day, preferably 10 μg to 120 mg/day, more preferably 25 μg to 120 mg/day, in total.

miRNA can be obtained by preparing a partially double-stranded RNA as a precursor of miRNA (pri-miRNA), and digesting it with a Dicer enzyme. As the Dicer enzyme, commercially available enzymes can be used. The double-stranded RNA can be prepared by, for example, a RNA polymerase reaction using a double-stranded DNA having a complementary sequence as a template. The double-stranded DNA can be prepared by amplification based on PCR using a chromosomal DNA of mammal as a template and primers designed so as to be able to amplify the sequence of miRNA.

miRNA can be obtained by digesting the double-stranded RNA obtained as described above with a Dicer enzyme or the like.

Further, miRNA can also be prepared by chemical synthesis. That is, miRNA can be obtained by synthesizing a sense strand and an antisense strand and annealing them.

Further, a double-stranded RNA that allows generation of a target miRNA by means of an endogenous Dicer enzyme of mammal may be added to the composition for oral ingestion.

When the composition for oral ingestion of the present invention is a pharmaceutical agent, the composition can be prepared by combining a miRNA with pharmaceutically acceptable carriers for oral administration. The form of the pharmaceutical preparation is not particularly limited, and examples include tablet, pill, powder, solution, suspension, emulsion, granule, capsule, syrup, and so forth. For the formulation, additives widely used for usual pharmaceutical agents as pharmaceutical carriers for oral administration such as excipients, binders, disintegrating agents, lubricants, stabilizers, corrigents, diluents, and surfactants can be used. Further, unless the effect of the present invention is degraded, miRNA may be used together with another drug having an immunoregulatory action.

Although amount of miRNA contained in the pharmaceutical agent is not particularly limited, it is, for example, 2 μg/kg to 2 mg/kg, preferably 4 μg/kg to 2 mg/kg, more preferably 10 μg/kg to 2 mg/kg, in total.

When the composition for oral ingestion is a foodstuff, it may be for any of various uses utilizing an immunostimulating action. Examples of the use include, for example, uses as foodstuffs suitable for persons showing reduced resistance, uses as foodstuffs or drinks useful for reduction and elimination of risk factors of various diseases caused by immune depression, and so forth.

The foodstuffs or drinks of the present invention can be marketed as foodstuffs attached with an indication describing that the foodstuffs are used for immunoregulation.

The aforementioned term “indication” includes all actions for informing consumers the aforementioned use, and any indications reminding or analogizing the aforementioned use fall within the scope of the “indication” of the present invention regardless of purpose, content, objective article, medium etc. of the indication. However, the indication is preferably made with an expression that allows consumers to directly recognize the aforementioned use. Specific examples include actions of indicating the aforementioned use on goods or packages of goods relating to the foodstuff of the present invention, actions of assigning, delivering, displaying for the purpose of assigning or delivering or importing such goods or packages of goods on which the aforementioned use is indicated, displaying or distributing advertisements, price lists or business papers relating the goods, or providing information including those as contents with indicating the aforementioned use by an electromagnetic method (Internet etc.) and so forth.

The indication is preferably an indication approved by the administration etc. (for example, an indication in a form based on an approval, which is qualified on the basis of any of various legal systems provided by the administration), and it is particularly preferably an indication on advertisement materials at the sales spots such as packages, containers, catalogs, pamphlets and POPs, others documents and so forth.

Examples of the indication further include, for example, indications as health food, functional food, enteric nutritive food, food for special dietary uses, food with nutrient function claims, quasi-drug and so forth as well as indications approved by the Ministry of Health, Labor and Welfare, for example, indications approved on the basis of the system of food for specified health uses and similar systems. Examples of the latter include indications as food for specified health uses, indications as food for specified health uses with qualified health claims, indications of influence on body structures and functions, indications of reduction of disease risk claims and so forth, and more precisely, typical examples include indications as food for specified health uses (especially indications of use for health) provided in the enforcement regulations of Health Promotion Law (Japan Ministry of Health, Labor and Welfare, Ministerial ordinance No. 86, Apr. 30, 2003) and similar indications.

### EXAMPLES

Hereafter, the present invention will be further specifically explained with reference to examples. However, the present invention is not limited to the following examples.

### Example 1

### Analysis of miRNAs in Breast Milk

Human breast milk was centrifuged at 2,000×g for 10 minutes to remove cells and large precipitates, and the supernatant except for the lipids constituting a surface layer was further centrifuged at 12,000×g for 30 minutes to remove cell debris and small dusts. Total RNA was extracted from the supernatant using the mirVana miRNA isolation kit according to the manufacturer's protocol. Extraction of RNAs from serum was performed in the same manner as that used for the breast milk.

The extracted RNAs were analyzed by using a bioanalyzer. Although a considerable amount of RNAs were contained in breast milk, ribosomal RNAs (18S rRNA, 28S rRNA) were scarcely contained, or were not contained at all.

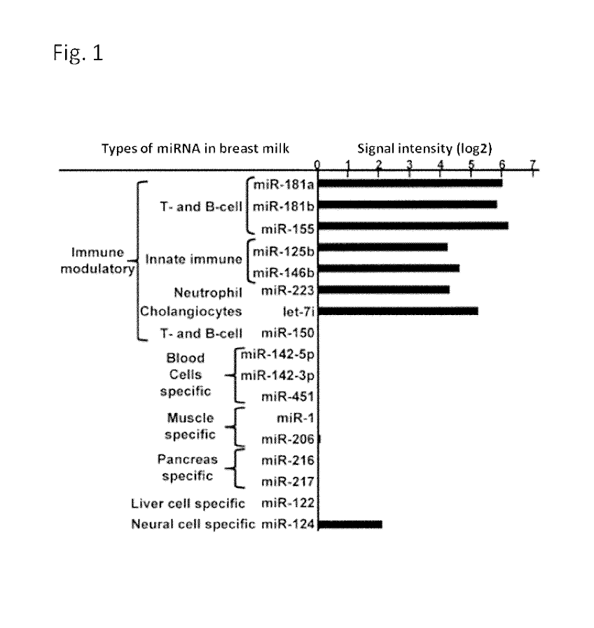

miRNAs were detected by using a microarray analysis system (one produced by Agilent Technologies was used). Expression level of miRNAs was analyzed by using GeneSpring GX11.0 (produced by Agilent Technologies). The results are shown in FIG. 1. As a result, miR-181a, miR-181b, miR-155, miR-125b, miR-146b, miR-223, and let-7i were detected in marked level. miR-150, which controls T cells and B cells, could not be detected. Further, a plurality of organ-specific miRNAs such as miR-122 (liver), miR-216, miR-217 (pancreas), miR-142-5p, and miR-142-3p (hematopoietic cell) could hardly be detected. Furthermore, miR-124 (brain) was detected in a small amount.

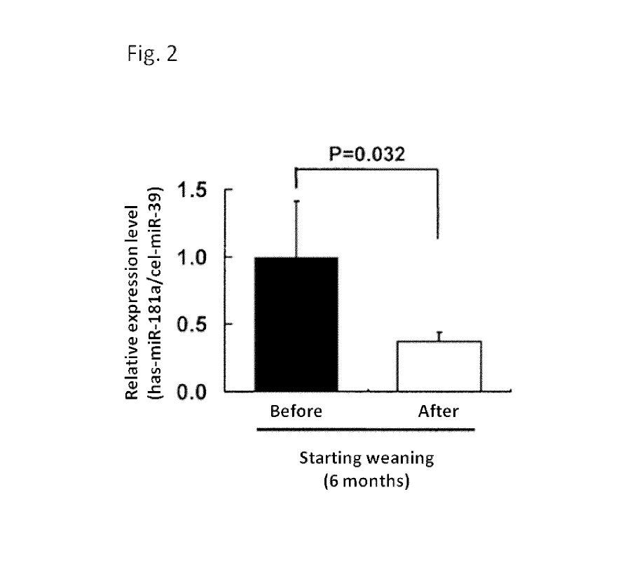

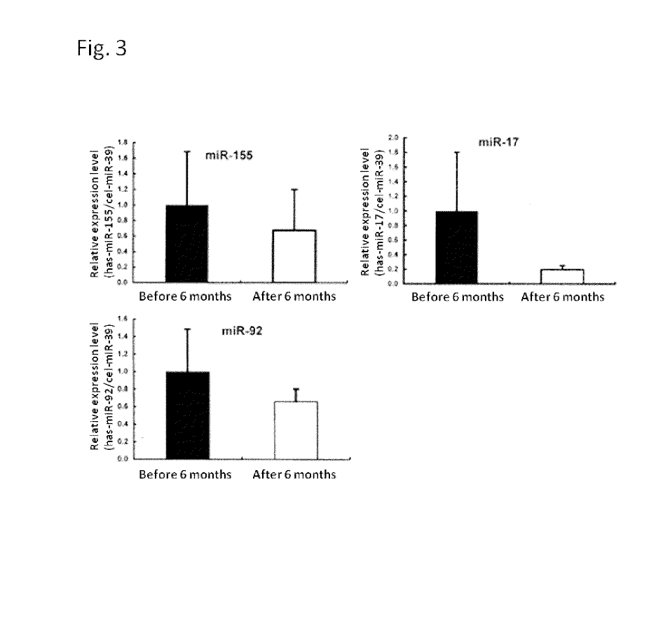

The results of comparison of miR-181a levels analyzed by quatitative RT-PCR in breast milk for first six months after the birth (n=5) and next six months (n=13) are shown as FIG. 2. The results of similar analyses conducted for miR-155, miR-17 and miR-92a are also shown in FIG. 3. In order to normalize the variations among the samples induced by the RNA isolation process, denatured cel-miR-39 (synthesized by Qiagen), which is a synthesized miRNA of a nematode (Caenorhabditis elegans), was added to the samples (at an oligonucleotide amount of 25 fmol in the total volume of 5 ml), and the amounts of miRNAs are shown as relative amounts based on the cel-miR-39 amount (the same shall apply to the following experiments).

As a result, the amount of miR-181a was larger in the milk of the first six months after the birth as compared to that in the milk of the six months thereafter (FIG. 2). Similar tendencies were also observed for miR-155, miR-17, and miR-92a (FIG. 3).

As the primers for RT-PCR, those produced by Applied Biosystems and identified by the following Assay IDs were used.

miR-181a: 000480

miR-155: 002623

miR-17: 002308

miR-92a: 000431

Cel-miR-39: 000200

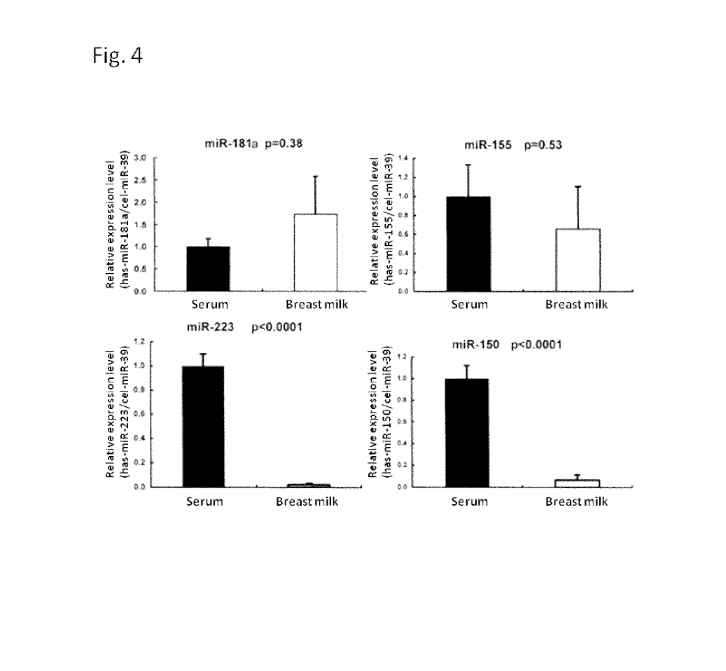

The results of comparison of immunity-related miRNA levels in breast milk and serum of seven healthy humans within 6 months post-partum are shown in FIG. 4 (breast milk: n=5, serum: n=6). The miRNA profiles in the breast milk are different from those in the serum. For example, miR-223, which is miRNA that controls granulocytes, existed at the highest level in normal human serum and plasma, whereas the expression amount thereof in the breast milk was extremely very lower as compared to that in the serum. Further, miR-146b which does not abundantly exist in the serum abundantly existed in the breast milk.

On the other hand, miR-181 and miR-155 abundantly existed in the breast milk at expression amounts comparable to those observed in the serum. It is interesting that a plurality of kinds of immunity-related miRNAs was highly expressed in the breast milk of post-partum six months, which is a stage before ingestion of baby food.

Intercellular transfer of miRNAs indicates that not only miRNAs control intracellular molecules, but also they are molecules playing a role in communication between cells like cytokines. The aforementioned results suggest that miRNAs are “genetic materials” that can be transferred from a mother to a child. It is calculated that about 0.15 pg/L/day (1.3×10⁷ copies/L/day) of miR-181 is ingested by an infant via breast milk.

In addition, it was found that miRNA profiles in breast milks of different mothers were similar, as a result of a cluster analysis.

### Example 2

### Physicochemical Properties of miRNA

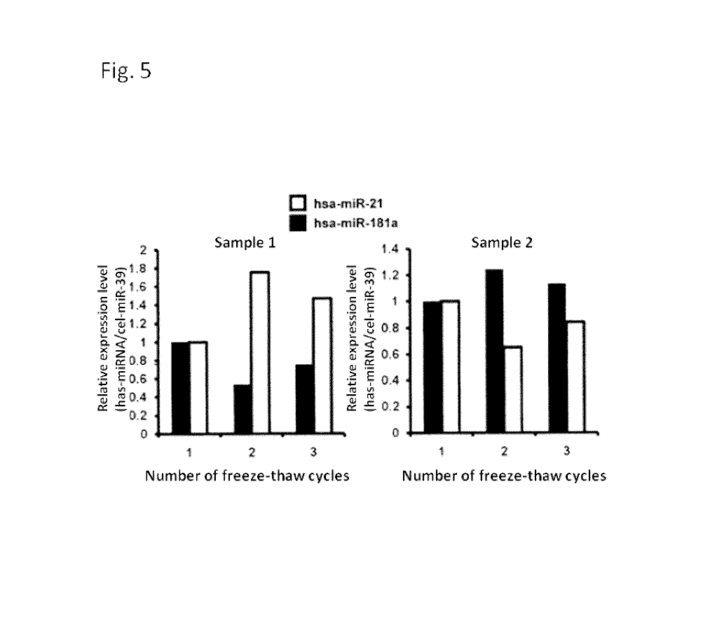

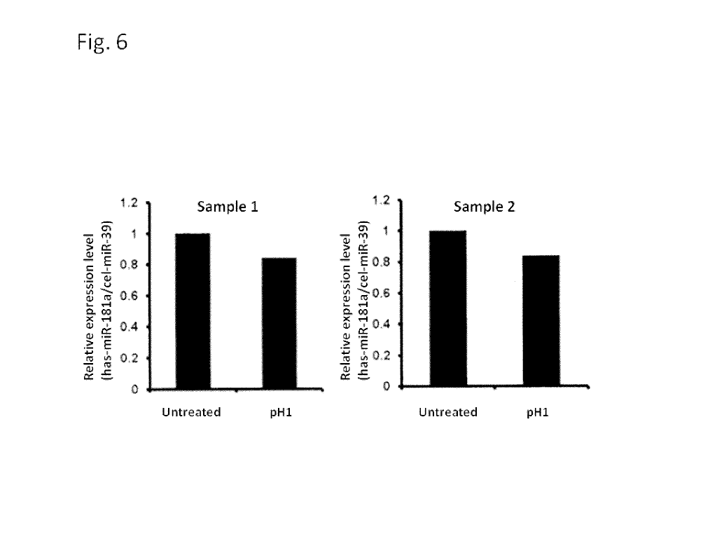

Breast milk was left standing at room temperature for 24 hours, or repeatedly subjected to freezing (−20° C.) and thawing up to 3 times. The levels of miRNAs (miR-21, miR-181a) were measured by TaqMan qRT-PCR. The results are shown in FIG. 5. Further, breast milk was treated in a low pH solution (pH 1) for 3 hours, and the miRNA level (miR-181a) was measured by TaqMan qRT-PCR before and after the treatment. The results are shown in FIG. 6.

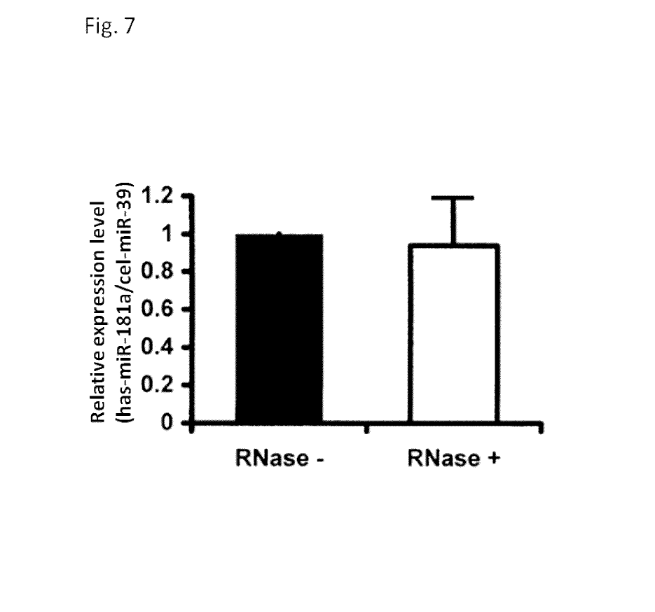

Further, to breast milk, an RNase A/T solution (mixed solution of RNase A (500 U/ml) and RNase T1 (20,000 U/ml), produced by Ambion) was added in a volume of 2% of the breast milk, the mixture was treated at 37° C. for 3 hours, and the miRNA level (miR-181a) was measured by TaqMan qRT-PCR before and after the treatment. The results are shown in FIG. 7.

As the primers for TaqMan qRT-PCR, those produced by Applied Biosystems and identified by the following Assay IDs were used.

miR-181a: 000480

miR-21: 000397

Cel-miR-39: 000200

It was demonstrated that miRNAs were relatively stable to freeze-thaw, low pH, and RNases.

### Example 3

### Identification of Diet or Substance Providing Production of Milk Having Immunoregulatory Action

SD rats at pregnancy day 9 to 10 were purchased, and a suspension of a Bifidobacterium bacteria, Bifidobacterium longum BB536 (ATCC BAA-999) in PBS (phosphate buffered saline) (1×10⁹ cfu/ml) was orally administered to the rats in a test group (n=3) everyday in a volume of 1 ml/day per rat in the period of pregnancy days 13 to 20.

Further, as a control group (n=3), PBS was administered everyday in a volume of 1 ml per rat. The B. longum ATCC BAA-999 strain can be purchased from American Type Culture Collection (Address: 12301 Parklawn Drive, Rockville, Md. 20852, United States of America).

All the rats gave birth on pregnancy day 21, and they were milked under anesthesia with ether on the first day after the birth. The obtained colostrum sample was centrifuged twice at 1,200×g and 4° C. for 10 minutes to remove the lipid layer and cell debris.

Then, the supernatant was centrifuged at 21,500×g and 4° C. for 40 minutes, and further centrifuged for 1 hour under the same conditions to remove the casein fraction and thereby obtain milk serum. Total RNA was obtained from the obtained milk serum sample by using miRNeasy Mini Kit (produced by Qiagen).

By using 100 ng of the obtained RNA sample, miRNAs were detected in a conventional manner using a microarray analysis system (produced by Agilent Technologies). The results were analyzed by using GeneSpring GX11.0 (produced by Agilent Technologies).

When statistical analysis of the microarray data was conducted by using GeneSpring GX11.0, it was found that the number of types of the microRNAs of which expression was confirmed in the test group and the control group in which they were detected was 155 in total. Such microRNAs are as follows. In addition, miR-150 was not detected.

MicroRNAs of which expression was confirmed in the test group and the control group, 155 types:

miR-16, miR-17-5p, miR-18 (miR-18a), miR-19 (miR-19b), miR-20 (miR-20a), miR-21, miR-23 (miR-23a), miR-27 (miR-27a, miR-27b), miR-29 (miR-29a, miR-29b, miR-29c, miR-29c*), miR-30 (miR-30a, miR-30c, miR-30d, miR-30e*), miR-33, miR-34b, miR-92a, miR-93, miR-100, miR-101 (miR-101a, miR-101b), miR-106b, miR-130b, miR-140*, miR-141, miR-143, miR-146a, miR-185, miR-186, miR-192, miR-193, miR-195, miR-200a, miR-205, miR-218, miR-219-5p, miR-221, miR-301a, miR-322, miR-340-5p, miR-361, miR-429, miR-455, miR-466b, miR-497, miR-500, miR-503, miR-532-5p, miR-542-3p

let-7a, let-7a*, let-7b, let-7c, let-7d, le-7e, let-7f, let-7i, miR-10 (miR-10a-5p, miR-10b), miR-15 (miR-15b), miR-19 (miR-19a), miR-20 (miR-20a*), miR-22, miR-23 (miR-23b), miR-24, miR-25, miR-26 (miR-26a, miR-26b), miR-28, miR-30 (miR-30a*, miR-30b-5p, miR-30c-1*, miR-30c-2*, miR-30e), miR-31, miR-34 (miR-34a), miR-96, miR-98, miR-99 (miR-99a, miR-99b), miR-103, miR-107, miR-125 (miR-125a-3p, miR-125a-5p, miR-125b-3p, miR-125b-5p), miR-128, miR-130 (miR-130a), miR-133 (miR-133a, miR-133b), miR-134, miR-139 (miR-139-3p), miR-140, miR-146 (miR-146b), miR-148 (miR-148b-3p), miR-151, miR-152, miR-181 (miR-181a, miR-181a*, miR-181b, miR-181c, miR-181d), miR-182, miR-183, miR-188, miR-196 (miR-196c), miR-199 (miR-199a-3p), miR-200 (miR-200b, miR-200c), miR-203, miR-204, miR-206, miR-210, miR-212, miR-214, miR-222, miR-223, miR-290, miR-291 (miR-291a-5p), miR-292 (miR-292-5p), miR-294, miR-296 (miR-296*), miR-320, miR-324 (miR-324-3p, miR-324-5p), miR-327, miR-328, miR-331, miR-340 (miR-340-3p), miR-341, miR-342 (miR-342-3p), miR-345 (miR-345-5p), miR-347, miR-352, miR-365, miR-370, miR-375, miR-378 (miR-378, miR-378*), miR-425, miR-465, miR-483, miR-484, miR-494, miR-542 (miR-542-5p), miR-652, miR-672, miR-685, miR-760 (miR-760-3p), miR-872, miR-874, miR-1224

The miRNAs listed with parenthesized indications following the miR-No. have subtypes, and subtypes indicated in the parentheses actually expressed.

Further, when expression amounts of the aforementioned microRNAs in the Bifidobacterium bacteria BB 536-administered group and the control group were statistically compared by using the Mann-Whitney U-test, it was found that the following 52 types of microRNAs increased in the Bifidobacterium bacteria BB 536-administered group at a probability level of less than 5%. Magnitudes of variation in expression of the miRNAs are shown in Table 11.

MicroRNAs of which increase was confirmed in the Bifidobacterium bacteria BB 536-administered group, 52 types:

miR-16, miR-17-5p, miR-18 (miR-18a), miR-19 (miR-19b), miR-20 (miR-20a), miR-21, miR-23 (miR-23a), miR-27 (miR-27a, miR-27b), miR-29 (miR-29a, miR-29b, miR-29c, miR-29c*), miR-30 (miR-30a, miR-30c, miR-30d, miR-30e*), miR-33, miR-34b, miR-92a, miR-93, miR-100, miR-101 (miR-101a, miR-101b), miR-106b, miR-130b, miR-140*, miR-141, miR-143, miR-146a, miR-185, miR-186, miR-192, miR-193, miR-195, miR-200a, miR-205, miR-218, miR-219-5p, miR-221, miR-301a, miR-322, miR-340-5p, miR-361, miR-429, miR-455, miR-466b, miR-497, miR-500, miR-503, miR-532-5p, miR-542-3p

TABLE 11

| | Mann-Whitney U test | | | |
|---|---|---|---|---|
| | Systematic name | p-Value | Regulation | Magnitude of variation |
| 1 | rno-miR-16 | 0.049535 | up | 1.67 |
| 2 | rno-miR-17-5p | 0.049535 | up | 1.83 |
| 3 | rno-miR-18a | 0.049535 | up | 2.03 |
| 4 | rno-miR-19b | 0.049535 | up | 1.64 |
| 5 | rno-miR-20a | 0.049535 | up | 2.04 |
| 6 | rno-miR-21 | 0.049535 | up | 1.92 |
| 7 | rno-miR-23a | 0.049535 | up | 1.68 |
| 8 | rno-miR-27a | 0.049535 | up | 1.64 |
| 9 | rno-miR-27b | 0.049535 | up | 1.98 |
| 10 | rno-miR-29a | 0.049535 | up | 1.53 |
| 11 | rno-miR-29b | 0.049535 | up | 1.92 |
| 12 | rno-miR-29c | 0.049535 | up | 1.64 |
| 13 | rno-miR-29c* | 0.049535 | up | 1.72 |
| 14 | rno-miR-30a | 0.049535 | up | 1.70 |
| 15 | rno-miR-30c | 0.049535 | up | 1.94 |
| 16 | rno-miR-30d | 0.049535 | up | 1.50 |
| 17 | rno-miR-30e* | 0.049535 | up | 2.01 |
| 18 | rno-miR-33 | 0.036904 | up | 2.53 |
| 19 | rno-miR-34b | 0.049535 | up | 3.02 |
| 20 | rno-miR-92a | 0.049535 | up | 2.09 |
| 21 | rno-miR-93 | 0.049535 | up | 1.70 |
| 22 | rno-miR-100 | 0.049535 | up | 2.08 |
| 23 | rno-miR-101a | 0.049535 | up | 2.81 |
| 24 | rno-miR-101b | 0.049535 | up | 1.97 |
| 25 | rno-miR-106b | 0.049535 | up | 1.74 |
| 26 | rno-miR-130b | 0.046302 | up | 4.83 |
| 27 | rno-miR-140* | 0.049535 | up | 1.83 |
| 28 | rno-miR-141 | 0.049535 | up | 1.76 |
| 29 | rno-miR-143 | 0.049535 | up | 2.16 |
| 30 | rno-miR-146a | 0.049535 | up | 1.95 |
| 31 | rno-miR-185 | 0.049535 | up | 1.74 |
| 32 | rno-miR-186 | 0.049535 | up | 1.70 |
| 33 | rno-miR-192 | 0.049535 | up | 2.37 |
| 34 | rno-miR-193 | 0.049535 | up | 2.10 |
| 35 | rno-miR-195 | 0.049535 | up | 2.37 |
| 36 | rno-miR-200a | 0.049535 | up | 1.88 |
| 37 | rno-miR-205 | 0.049535 | up | 1.47 |
| 38 | rno-miR-218 | 0.049535 | up | 1.91 |
| 39 | rno-miR-219-5p | 0.049535 | up | 1.73 |
| 40 | rno-miR-221 | 0.049535 | up | 2.02 |
| 41 | rno-miR-301a | 0.049535 | up | 1.59 |
| 42 | rno-miR-322 | 0.049535 | up | 1.72 |
| 43 | rno-miR-340-5p | 0.049535 | up | 3.12 |
| 44 | rno-miR-361 | 0.049535 | up | 1.83 |
| 45 | rno-miR-429 | 0.049535 | up | 1.52 |
| 46 | rno-miR-455 | 0.049535 | up | 2.33 |
| 47 | rno-miR-466b | 0.049535 | up | 1.55 |
| 48 | rno-miR-497 | 0.049535 | up | 2.41 |
| 49 | rno-miR-500 | 0.049535 | up | 1.91 |
| 50 | rno-miR-503 | 0.049535 | up | 6.91 |
| 51 | rno-miR-532-5p | 0.049535 | up | 2.78 |
| 52 | rno-miR-542-3p | 0.049535 | up | 3.13 |

As seen from the results shown in Table 11, it was found that the magnitudes of the variation observed for all the 52 types of the microRNAs of which increases were confirmed were 1.2 times or larger.

That is, it was found that the Bifidobacterium bacteria BB536 strain could be screened for as a diet or a substance providing production of milk having an immunoregulatory action on the basis of detection of these 52 types of microRNAs.

### Example 4

### Detection of microRNAs Expressed in Rat Colostrum

Three F344 rats on pregnancy day 14 were purchased. All the purchased rats gave birth on pregnancy day 21, and they were milked under anesthesia with ether on the second day after the birth to collect colostrum.

Each colostrum sample was centrifuged twice at 1,200×g and 4° C. for 10 minutes to remove the lipid layer and cell debris.

Then, the supernatant was centrifuged at 21,500×g and 4° C. for 40 minutes, and further centrifuged for 1 hour under the same conditions to remove the casein fraction and thereby obtain milk serum.

Total RNA was obtained from the obtained milk serum sample by using miRNeasy Mini Kit (produced by Qiagen).

The obtained RNA sample in an amount of 100 ng was used in an experiment on a microarray (produced by Agilent Technologies) in a conventional manner. The results of the microarray experiment were analyzed by using GeneSpring GX11.0 (produced by Agilent Technologies).

As a result, it was confirmed that four kinds of microRNAs (miR-193*, miR-409-3p, miR-664, miR-877) were expressed in addition to the 155 kinds of microRNAs confirmed in Example 3.

### Example 5

### Detection of microRNAs Expressed in Bovine Colostrum

Five samples of milk of Holstein cows in the period of the post-partum days 1 to 3 were prepared as colostrum samples. Further, five samples of milk of Holstein cows in the period from the post-partum day 8 to 8 months were prepared as normal milk samples.

Each of the milk samples (colostrum and normal milk) was centrifuged twice at 1,200×g and 4° C. for 10 minutes to remove the lipid layer and cell debris.

Then, the supernatant was centrifuged at 21,500×g and 4° C. for 40 minutes, and further centrifuged for 1 hour under the same conditions to remove the casein fraction and thereby obtain milk serum.

Total RNA was obtained from the obtained milk serum sample by using miRNeasy Mini Kit (produced by Qiagen).

The obtained RNA sample in an amount of 20 ng was used in an experiment on a microarray (produced by Agilent Technologies) in a conventional manner. The results of the microarray experiment were analyzed by using GeneSpring GX11.0 (produced by Agilent Technologies).

As a result, expression of 102 kinds in total of miRNAs was confirmed in the colostrum samples and the normal milk samples. In particular, among the 102 kinds of miRNAs, expression of 49 kinds of miRNAs was confirmed only in the colostrum.

The 49 kinds of microRNAs of which expression was confirmed only in the colostrum samples are mentioned below.

MicroRNAs of which expression was confirmed only in the colostrums, 49 types:

let-7d, let-7i, miR-15a, miR-15b, miR-16b, miR-17-3p, miR-19b, miR-21, miR-23b-3p, miR-24-3p, miR-26b, miR-27b, miR-30a-5p, miR-30c, miR-30f, miR-34a, miR-99a, miR-106, miR-106b, miR-107, miR-125b, miR-126, miR-129-3p, miR-130a, miR-130b, miR-140, miR-155, miR-181b, miR-184, miR-193a-3p, miR-193a-5p, miR-196a, miR-210, miR-222, miR-223, miR-338, miR-361, miR-362-5p, miR-370, miR-429, miR-452, miR-486, miR-500, miR-532, miR-584, miR-708, miR-877, miR-1300b, miR-1307

### INDUSTRIAL APPLICABILITY

According to the present invention, a diet or a substance contained therein providing production of milk having an immunoregulatory action can be screened for. The present invention also provides a method for producing dairy products having an immunoregulatory action. The composition for oral ingestion of the present invention has an immunostimulating action, and is especially useful for infants.

What is claimed is:

1. A method for producing milk or dairy products having an immunoregulatory action, which comprises:
providing a mammal except for human with a diet or a substance which increases or decreases the amount of microRNA in milk of the mammal, thereby allowing production of milk which has an immunoregulatory action, and
collecting milk from the mammal.

2. The method according to claim 1, wherein the immunoregulatory action is an immunostimulating action, and wherein the diet or substance increases the amount of the microRNA.

3. The method according to claim 1, wherein the immunoregulatory action is an immunosuppressive action, and wherein the diet or substance decreases the amount of the microRNA.

4. The method according to claim 2, wherein the diet or the substance has been designed by the process comprising:
allowing a mammal to ingest a diet,
measuring microRNA profiles in the milk of the mammal and microRNA profiles in serum or plasma before and after ingestion of the diet,
comparing microRNA profiles in the milk and in serum or plasma observed before and after ingestion of the diet, and
identifying the diet or a substance contained in the diet as a diet or a substance providing production of milk having an immunostimulating action when the amount of microRNA which presents in both the milk and the serum or plasma observed in the milk after the ingestion of the diet is 1.2 times or more as high as that observed in the milk before the ingestion of the diet.

5. The method according to claim 2, further comprising screening for a diet or a substance providing production of milk having an immunostimulating action, which comprises:
allowing a mammal to ingest a diet,
measuring microRNA profiles in the milk of the mammal and microRNA profiles in serum or plasma before and after ingestion of the diet,
comparing microRNA profiles in the milk and in serum or plasma observed before and after ingestion of the diet, and
identifying the diet or a substance contained in the diet as a diet or a substance providing production of milk having an immunostimulating action when the amount of microRNA which presents in both the milk and the serum or plasma observed in the milk after the ingestion of the diet is 1.2 times or more as high as that observed in the milk before the ingestion of the diet.

6. The method according to claim 3, wherein the diet or the substance has been designed by the process comprising:
allowing a mammal to ingest a diet,
measuring microRNA profiles in the milk of the mammal and microRNA profiles in serum or plasma before and after ingestion of the diet,
comparing microRNA profiles in the milk and in serum or plasma observed before and after ingestion of the diet, and
identifying the diet or a substance contained in the diet as a diet or a substance providing production of milk having an immunosuppressive action when the amount of microRNA which presents in both the milk and the serum or plasma observed in the milk after the ingestion of the diet is 0.8 time or less as low as that observed in the milk before the ingestion of the diet.

7. The method according to claim 3, further comprising screening for a diet or a substance providing production of milk having an immunosuppressive action, which comprises:
allowing a mammal to ingest a diet,
measuring microRNA profiles in the milk of the mammal and microRNA profiles in serum or plasma before and after ingestion of the diet,
comparing microRNA profiles in the milk and in serum or plasma observed before and after ingestion of the diet, and
identifying the diet or a substance contained in the diet as a diet or a substance providing production of milk having an immunosuppressive action when the amount of microRNA which presents in both the milk and the serum or plasma observed in the milk after the ingestion of the diet is 0.8 time or less as low as that observed in the milk before the ingestion of the diet.

8. The method according to claim 1, wherein the microRNA is selected from the group consisting of miR-10, miR-15, miR-16, miR-17, miR-18, miR-19, miR-20, miR-21, miR-22, miR-23, miR-24, miR-25, miR-26, miR-27, miR-28, miR-29, miR-30, miR-31, miR-33, miR-34, miR-92, miR-93, miR-96, miR-98, miR-99, miR-100, miR-101, miR-103, miR-106, miR-107, miR-125, miR-126, miR-128, miR-129, miR-130, miR-133, miR-134, miR-139, miR-140, miR-141, miR-143, miR-146, miR-148, miR-151, miR-152, miR-155, miR-181, miR-182, miR-183, miR-184, miR-185, miR-186, miR-188, miR-192, miR-193, miR-195, miR-196, miR-199, miR-200, miR-203, miR-204, miR-205, miR-206, miR-210, miR-212, miR-214, miR-218, miR-219, miR-221, miR-222, miR-223, miR-290, miR-291, miR-292, miR-294, miR-296, miR-301, miR-320, miR-322, miR-324, miR-327, miR-328, miR-331, miR-338, miR-340, miR-341, miR-342, miR-345, miR-347, miR-352, miR-361, miR-362, miR-365, miR-370, miR-375, miR-378, miR-409, miR-425, miR-429, miR-452, miR-455, miR-465, miR-466, miR-483, miR-484, miR-486, miR-494, miR-497, miR-500, miR-503, miR-532, miR-542, miR-584, miR-652, miR-664, miR-672, miR-685, miR-708, miR-760, miR-872, miR-874, miR-877, miR-1224, miR-1300, miR-1307, let-7a, let-7b, let-7c, let-7d, let-7e, let-7f, and let-7i.

9. The method according to claim 1, wherein the microRNA is selected from the group consisting of miR-15, miR-16, miR-17, miR-18, miR-19, miR-20, miR-21, miR-23, miR-24, miR-26, miR-27, miR-29, miR-30, miR-33, miR-34, miR-92, miR-93, miR-99, miR-100, miR-101, miR-106, miR-107, miR-125, miR-130, miR-140, miR-141, miR-143, miR-146, miR-155, miR-181, miR-185, miR-186, miR-192, miR-193, miR-195, miR-200, miR-205, miR-210, miR-218, miR-219, miR-221, miR-222, miR-223, miR-301, miR-322, miR-340, miR-361, miR-370, miR-429, miR-455, miR-466, miR-497, miR-500, miR-503, miR-532, miR-542, let-7d, and let-7i.

10. The method according to claim 1, wherein the microRNA is selected from the group consisting of miR-15, miR-16, miR-19, miR-21, miR-23, miR-24, miR-26, miR-27, miR-30, miR-34, miR-99, miR-106, miR-107, miR-125, miR-130, miR-140, miR-181, miR-193, miR-210, miR-222, miR-223, miR-361, miR-370, miR-429, miR-500, miR-532, let-7d, and let-7i.

11. A method for immunostimulation in a subject, comprising orally administering a composition to the subject, wherein the composition comprises microRNA having an immunostimulating action.

12. The method according to claim 11, wherein content of the microRNA in the composition is 10 to 10,000 ng/ml.

13. The method according to claim 11, wherein the microRNA is selected from the group consisting of miR-10, miR-15, miR-16, miR-17, miR-18, miR-19, miR-20, miR-21, miR-22, miR-23, miR-24, miR-25, miR-26, miR-27, miR-28, miR-29, miR-30, miR-31, miR-33, miR-34, miR-92, miR-93, miR-96, miR-98, miR-99, miR-100, miR-101, miR-103, miR-106, miR-107, miR-125, miR-126, miR-128, miR-129, miR-130, miR-133, miR-134, miR-139, miR-140, miR-141, miR-143, miR-146, miR-148, miR-151, miR-152, miR-155, miR-181, miR-182, miR-183, miR-184, miR-185, miR-186, miR-188, miR-192, miR-193, miR-195, miR-196, miR-199, miR-200, miR-203, miR-204, miR-205, miR-206, miR-210, miR-212, miR-214, miR-218, miR-219, miR-221, miR-222, miR-223, miR-290, miR-291, miR-292, miR-294, miR-296, miR-301, miR-320, miR-322, miR-324, miR-327, miR-328, miR-331, miR-338, miR-340, miR-341, miR-342, miR-345, miR-347, miR-352, miR-361, miR-362, miR-365, miR-370, miR-375, miR-378, miR-409, miR-425, miR-429, miR-452, miR-455, miR-465, miR-466, miR-483, miR-484, miR-486, miR-494, miR-497, miR-500, miR-503, miR-532, miR-542, miR-584, miR-652, miR-664, miR-672, miR-685, miR-708, miR-760, miR-872, miR-874, miR-877, miR-1224, miR-1300, miR-1307, let-7a, let-7b, let-7c, let-7d, let-7e, let-7f, and let-7i.

14. The method according to claim 11, wherein the microRNA is selected from the group consisting of miR-15, miR-16, miR-17, miR-18, miR-19, miR-20, miR-21, miR-23, miR-24, miR-26, miR-27, miR-29, miR-30, miR-33, miR-34, miR-92, miR-93, miR-99, miR-100, miR-101, miR-106, miR-107, miR-125, miR-130, miR-140, miR-141, miR-143, miR-146, miR-155, miR-181, miR-185, miR-186, miR-192, miR-193, miR-195, miR-200, miR-205, miR-210, miR-218, miR-219, miR-221, miR-222, miR-223, miR-301, miR-322, miR-340, miR-361, miR-370, miR-429, miR-455, miR-466, miR-497, miR-500, miR-503, miR-532, miR-542, let-7d, and let-7i.

15. The method according to claim 11, wherein the microRNA is selected from the group consisting of miR-15, miR-16, miR-19, miR-21, miR-23, miR-24, miR-26, miR-27, miR-30, miR-34, miR-99, miR-106, miR-107, miR-125, miR-130, miR-140, miR-181, miR-193, miR-210, miR-222, miR-223, miR-361, miR-370, miR-429, miR-500, miR-532, let-7d, and let-7i.

16. The method according to claim 11, wherein the composition is a foodstuff for infants or a foodstuff for little children.

17. The method according to claim 16, wherein the foodstuff for infants or foodstuff for little children is infant formula or follow-up formula.


SEQUENCE LISTING (504 sequences)

SEQ ID NO: 1 - RNA, 65 nt, Homo sapiens
cuguuaaugcuaaucgugauagggguuuuugccuccaacugacuccuacauauuagcauuaacag

SEQ ID NO: 2 - RNA, 23 nt, Homo sapiens
uuaaugcuaaucgugauaggggu

SEQ ID NO: 3 - RNA, 63 nt, Bos taurus
uguuaaugcuaaucgugauagggguuuuuaccucggacugacuccuacauguuagcauuaaca

SEQ ID NO: 4 - RNA, 23 nt, Bos taurus
uuaaugcuaaucgugauaggggu

SEQ ID NO: 5 - RNA, 110 nt, Homo sapiens
ugaguuuugagguugcuucagugaacauucaacgcugucggugaguuuggaauuaaaaucaaaaccaucgaccguugauuguacccuauggcuaaccaucaucuacucca

SEQ ID NO: 6 - RNA, 110 nt, Homo sapiens
agaagggcuaucaggccagccuucagaggacuccaaggaacauucaacgcugucggugaguuugggauuugaaaaaaccacugaccguugacuguaccuugggguccuua

SEQ ID NO: 7 - RNA, 23 nt, Homo sapiens
aacauucaacgcugucggugagu

SEQ ID NO: 8 - RNA, 110 nt, Homo sapiens
ccugugcagagauuauuuuuuaaaaggucacaaucaacauucauugcugucgguggguugaacuguguggacaagcucacugaacaaugaaugcaacuguggccccgcuu

SEQ ID NO: 9 - RNA, 89 nt, Homo sapiens
cugauggcugcacucaacauucauugcugucgguggguuugagucugaaucaacucacugaucaaugaaugcaaacugcggaccaaaca

SEQ ID NO: 10 - RNA, 23 nt, Homo sapiens
aacauucauugcugucggugggu

SEQ ID NO: 11 - RNA, 110 nt, Bos taurus
ugccagggccaggacccagucuucagaggacuccaaggaacauucaacgcugucggugaguuugggauuugaaaaaaccaccgaccguugacuguaccuuggguuccuua

SEQ ID NO: 12 - RNA, 110 nt, Bos taurus
ugagcuccgagguugcuucagugaacauucaacgcugucggugaguuuggaauuaaaaaucaaaaccaucgaccguugauuguacccuauggccaaccaccaucuccacc

SEQ ID NO: 13 - RNA, 24 nt, Bos taurus
aacauucaacgcugucggugaguu

SEQ ID NO: 14 - RNA, 110 nt, Bos taurus
cuugggcagagguucuuucuuaaaaggucacaaucaacauucauugcugucgguggguugaacuguguggacaagcucacugaacaaugagugcaacuguggccccgcau

SEQ ID NO: 15 - RNA, 89 nt, Bos taurus
cugauggcugcacucaacauucauugcugucgguggguuugacuuugaaucaacucacugaucaaugaauguaaacugcggaccaaaca

SEQ ID NO: 16 - RNA, 24 nt, Bos taurus
aacauucauugcugucgguggguu

SEQ ID NO: 17 - RNA, 110 nt, Homo sapiens
ccuggccuccugcagugccacgcuccguguauuugacaagcugaguuggacacuccaugugguagagugucaguuugucaaauaccccaagugcggcacaugcuuaccag

SEQ ID NO: 18 - RNA, 22 nt, Homo sapiens
ugucaguuugucaaauacccca

SEQ ID NO: 19 - RNA, 108 nt, Bos taurus
cccagccuccugcagugccaugcuccguguauuugacaagcugaguuggacacuccauguaguagugucaguuugucaaauaccccaaguguggcauaugccuagcag

SEQ ID NO: 20 - RNA, 22 nt, Bos taurus
ugucaguuugucaaauacccca

SEQ ID NO: 21 - RNA, 84 nt, Homo sapiens
gucagaauaaugucaaagugcuuacagugcagguagugauaugugcaucuacugcagugaaggcacuuguagcauuauggugac

SEQ ID NO: 22 - RNA, 23 nt, Homo sapiens
caaagugcuuacagugcagguag

SEQ ID NO: 23 - RNA, 84 nt, Bos taurus; identical to SEQ ID NO: 21

SEQ ID NO: 24 - RNA, 24 nt, Bos taurus
caaagugcuuacagugcagguagu

SEQ ID NO: 25 - RNA, 20 nt, Bos taurus
acugcagugaaggcacuugu

SEQ ID NO: 26 - RNA, 78 nt, Homo sapiens
cuuucuacacagguugggaucgguugcaaugcuguguuucuguaugguauugcacuugucccggccuguugaguuugg

SEQ ID NO: 27 - RNA, 75 nt, Homo sapiens
ucaucccuggguggggauuuguugcauuacuuguguucuauauaaaguauugcacuugucccggccuguggaaga

SEQ ID NO: 28 - RNA, 22 nt, Homo sapiens
uauugcacuugucccggccugu

SEQ ID NO: 29 - RNA, 96 nt, Homo sapiens
cgggccccgggcgggcgggagggacgggacgcggugcaguguuguuuuuucccccgccaauauugcacucgucccggccuccggcccccccggccc

SEQ ID NO: 30 - RNA, 22 nt, Homo sapiens
uauugcacucgucccggccucc

SEQ ID NO: 31 - RNA, 68 nt, Bos taurus
uggguggggauuuguugcacugcuuguguuauauauaaaguauugcacuugucccggccuguggaaga

SEQ ID NO: 32 - RNA, 22 nt, Bos taurus
uauugcacuugucccggccugu

SEQ ID NO: 33 - RNA, 78 nt, Bos taurus; identical to SEQ ID NO: 26

SEQ ID NO: 34 - RNA, 22 nt, Bos taurus
uauugcacuugucccggccugu

SEQ ID NO: 35 - RNA, 96 nt, Bos taurus
cgggccccgggcgggcgggagggacgggacgcggugcaguguuguucuuuccccugccaauauugcacucgucccggccuccggcccccucggccc

SEQ ID NO: 36 - RNA, 22 nt, Bos taurus
uauugcacucgucccggccucc

SEQ ID NO: 37 - RNA, 84 nt, Homo sapiens
cuggcugagguaguaguuugugcuguuggucggguugugacauugcccgcuguggagauaacugcgcaagcuacugccuugcua

SEQ ID NO: 38 - RNA, 22 nt, Homo sapiens
ugagguaguaguuugugcuguu

SEQ ID NO: 39 - RNA, 84 nt, Bos taurus; identical to SEQ ID NO: 37

SEQ ID NO: 40 - RNA, 22 nt, Bos taurus
ugagguaguaguuugugcuguu

SEQ ID NO: 41 - RNA, 86 nt, Homo sapiens
ugccagucucuaggucccugagacccuuuaaccugugaggacauccagggucacaggugagguucuugggagccuggcgucuggcc

SEQ ID NO: 42 - RNA, 24 nt, Homo sapiens
ucccugagacccuuuaaccuguga

SEQ ID NO: 43 - RNA, 22 nt, Homo sapiens
acaggugagguucuugggagcc

SEQ ID NO: 44 - RNA, 88 nt, Homo sapiens
ugcgcuccucucagucccugagacccuaacuugugauguuuaccguuuaaauccacggguuaggcucuugggagcugcgagucgugcu

SEQ ID NO: 45 - RNA, 89 nt, Homo sapiens
accagacuuuuccuagucccugagacccuaacuugugagguauuuuaguaacaucacaagucaggcucuugggaccuaggcggagggga

SEQ ID NO: 46 - RNA, 22 nt, Homo sapiens
ucccugagacccuaacuuguga

SEQ ID NO: 47 - RNA, 86 nt, Bos taurus
ugccggccucugcgucccugagacccuuuaaccugugaggacguccagggucacaggugagguucuugggagccuggcguccggcc

SEQ ID NO: 48 - RNA, 23 nt, Bos taurus
ucccugagacccuuuaaccugug

SEQ ID NO: 49 - RNA, 88 nt, Bos taurus
cgcgcgccucucaaucccugagacccuaacuugugauguuuaccguuuaaauccacggguuaggcucuugggagcugcgagucgcgcc

SEQ ID NO: 50 - RNA, 85 nt, Bos taurus
gacuuuuccuagucccugagacccuaacuugugagguauuuuaguagcaucacaagucaggcucuugggaccuaggcggagggga

SEQ ID NO: 51 - RNA, 22 nt, Bos taurus
ucccugagacccuaacuuguga

SEQ ID NO: 52 - RNA, 99 nt, Homo sapiens
ccgauguguauccucagcuuugagaacugaauuccauggguugugucagugucagaccucugaaauucaguucuucagcugggauaucucugucaucgu

SEQ ID NO: 53 - RNA, 22 nt, Homo sapiens
ugagaacugaauuccauggguu

SEQ ID NO: 54 - RNA, 73 nt, Homo sapiens
ccuggcacugagaacugaauuccauaggcugugagcucuagcaaugcccuguggacucaguucuggugcccgg

SEQ ID NO: 55 - RNA, 22 nt, Homo sapiens
ugagaacugaauuccauaggcu

SEQ ID NO: 56 - RNA, 22 nt, Homo sapiens
ugcccuguggacucaguucugg

SEQ ID NO: 57 - RNA, 99 nt, Bos taurus
cccauguguauccucagcuuugagaacugaauuccauagguugugucagugucagaccugugaaguuuaguucuuuagcugggauaucucuaucauccu

SEQ ID NO: 58 - RNA, 24 nt, Bos taurus
ugagaacugaauuccauagguugu

SEQ ID NO: 59 - RNA, 106 nt, Bos taurus
uaagagaacuuuggccaccuggcucugagaacugaauuccauaggcugugagcucuagcaaaugcccuagggacucaguucuggugcccggcugugcuacaccauc

SEQ ID NO: 60 - RNA, 24 nt, Bos taurus
ugagaacugaauuccauaggcugu

SEQ ID NO: 61 - RNA, 84 nt, Homo sapiens
cuccccauggcccugucucccaacccuuguaccagugcugggcucagacccugguacaggccugggggacagggaccuggggac

SEQ ID NO: 62 - RNA, 22 nt, Homo sapiens
ucucccaacccuuguaccagug

SEQ ID NO: 63 - RNA, 100 nt, Bos taurus
ccucucuccucacggcccugucucccaacccuuguaccagugugugucucagacccugguacagguacggggaggcagggaccugggggaucccagcagc

SEQ ID NO: 64 - RNA, 23 nt, Bos taurus
ucucccaacccuuguaccagugu

SEQ ID NO: 65 - RNA, 23 nt, Rattus norvegicus
caaagugcuuacagugcagguag

SEQ ID NO: 66 - RNA, 21 nt, Rattus norvegicus
uauugcacuugucccggccug

SEQ ID NO: 67 - RNA, 22 nt, Rattus norvegicus
ugagaacugaauuccauggguu

SEQ ID NO: 68 - RNA, 22 nt, Homo sapiens
uagcagcacguaaauauuggcg

SEQ ID NO: 69 - RNA, 22 nt, Rattus norvegicus
uagcagcacguaaauauuggcg

SEQ ID NO: 70 - RNA, 22 nt, Bos taurus
uagcagcacguaaauauuggug

SEQ ID NO: 71 - RNA, 23 nt, Homo sapiens
uaaggugcaucuagugcagauag

SEQ ID NO: 72 - RNA, 22 nt, Bos taurus
uaaggugcaucuagugcagaua

SEQ ID NO: 73 - RNA, 23 nt, Rattus norvegicus
uaaggugcaucuagugcagauag

SEQ ID NO: 74 - RNA, 23 nt, Homo sapiens
ugugcaaauccaugcaaaacuga

SEQ ID NO: 75 - RNA, 23 nt, Bos taurus
ugugcaaauccaugcaaaacuga

SEQ ID NO: 76 - RNA, 23 nt, Rattus norvegicus
ugugcaaauccaugcaaaacuga

SEQ ID NO: 77 - RNA, 23 nt, Homo sapiens
uaaagugcuuauagugcagguag

SEQ ID NO: 78 - RNA, 23 nt, Bos taurus
uaaagugcuuauagugcagguag

SEQ ID NO: 79 - RNA, 23 nt, Rattus norvegicus
uaaagugcuuauagugcagguag

SEQ ID NO: 80 - RNA, 22 nt, Homo sapiens
uagcuuaucagacugauguuga

SEQ ID NO: 81 - RNA, 24 nt, Bos taurus
uagcuuaucagacugauguugacu

SEQ ID NO: 82 - RNA, 22 nt, Rattus norvegicus
uagcuuaucagacugauguuga

SEQ ID NO: 83 - RNA, 21 nt, Homo sapiens
aucacauugccagggauuucc

SEQ ID NO: 84 - RNA, 22 nt, Bos taurus
aucacauugccagggauuucca

SEQ ID NO: 85 - RNA, 21 nt, Rattus norvegicus
aucacauugccagggauuucc

SEQ ID NO: 86 - RNA, 21 nt, Homo sapiens
uucacaguggcuaaguuccgc

SEQ ID NO: 87 - RNA, 21 nt, Rattus norvegicus
uucacaguggcuaaguuccgc

SEQ ID NO: 88 - RNA, 20 nt, Bos taurus
uucacaguggcuaaguuccg

SEQ ID NO: 89 - RNA, 22 nt, Bos taurus
agggcuuagcugcuugugagca

SEQ ID NO: 90 - RNA, 21 nt, Homo sapiens
uucacaguggcuaaguucugc

SEQ ID NO: 91 - RNA, 21 nt, Bos taurus
uucacaguggcuaaguucugc

SEQ ID NO: 92 - RNA, 21 nt, Rattus norvegicus
uucacaguggcuaaguucugc

SEQ ID NO: 93 - RNA, 22 nt, Homo sapiens
uagcaccaucugaaaucgguua

SEQ ID NO: 94 - RNA, 23 nt, Bos taurus
cuagcaccaucugaaaucgguua

SEQ ID NO: 95 - RNA, 22 nt, Rattus norvegicus
uagcaccaucugaaaucgguua

SEQ ID NO: 96 - RNA, 23 nt, Homo sapiens
uagcaccauuugaaaucaguguu

SEQ ID NO: 97 - RNA, 23 nt, Bos taurus
uagcaccauuugaaaucaguguu

SEQ ID NO: 98 - RNA, 23 nt, Rattus norvegicus
uagcaccauuugaaaucaguguu

SEQ ID NO: 99 - RNA, 22 nt, Homo sapiens
uagcaccauuugaaaucgguua

SEQ ID NO: 100 - RNA, 22 nt, Bos taurus
uagcaccauuugaaaucgguua

SEQ ID NO: 101 - RNA, 22 nt, Rattus norvegicus
uagcaccauuugaaaucgguua

SEQ ID NO: 102 - RNA, 22 nt, Homo sapiens
ugaccgauuucuccugguguuc

SEQ ID NO: 103 - RNA, 22 nt, Rattus norvegicus
ugaccgauuucuccugguguuc

SEQ ID NO: 104 - RNA, 22 nt, Homo sapiens
uguaaacauccucgacuggaag

SEQ ID NO: 105 - RNA, 24 nt, Bos taurus
uguaaacauccucgacuggaagcu

SEQ ID NO: 106 - RNA, 22 nt, Rattus norvegicus
uguaaacauccucgacuggaag

SEQ ID NO: 107 - RNA, 23 nt, Homo sapiens
uguaaacauccuacacucucagc

SEQ ID NO: 108 - RNA, 23 nt, Bos taurus
uguaaacauccuacacucucagc

SEQ ID NO: 109 - RNA, 23 nt, Rattus norvegicus
uguaaacauccuacacucucagc

SEQ ID NO: 110 - RNA, 22 nt, Homo sapiens
uguaaacauccccgacuggaag

SEQ ID NO: 111 - RNA, 24 nt, Bos taurus
uguaaacauccccgacuggaagcu

SEQ ID NO: 112 - RNA, 22 nt, Rattus norvegicus
uguaaacauccccgacuggaag

SEQ ID NO: 113 - RNA, 22 nt, Homo sapiens
cuuucagucggauguuuacagc

SEQ ID NO: 114 - RNA, 22 nt, Rattus norvegicus
cuuucagucggauguuuacagc

SEQ ID NO: 115 - RNA, 21 nt, Homo sapiens
gugcauuguaguugcauugca

SEQ ID NO: 116 - RNA, 21 nt, Bos taurus
gugcauuguaguugcauugca

SEQ ID NO: 117 - RNA, 21 nt, Rattus norvegicus
gugcauuguaguugcauugca

SEQ ID NO: 118 - RNA, 22 nt, Homo sapiens
caaucacuaacuccacugccau

SEQ ID NO: 119 - RNA, 22 nt, Bos taurus
aggcaguguaauuagcugauug

SEQ ID NO: 120 - RNA, 23 nt, Rattus norvegicus
uaggcaguguaauuagcugauug

SEQ ID NO: 121 - RNA, 23 nt, Homo sapiens
caaagugcuguucgugcagguag

SEQ ID NO: 122 - RNA, 22 nt, Bos taurus
caaagugcuguucgugcaggua

SEQ ID NO: 123 - RNA, 23 nt, Rattus norvegicus
caaagugcuguucgugcagguag

SEQ ID NO: 124 - RNA, 22 nt, Homo sapiens
aacccguagauccgaacuugug

SEQ ID NO: 125 - RNA, 22 nt, Bos taurus
aacccguagauccgaacuugug

SEQ ID NO: 126 - RNA, 22 nt, Rattus norvegicus
aacccguagauccgaacuugug

SEQ ID NO: 127 - RNA, 21 nt, Homo sapiens
uacaguacugugauaacugaa

SEQ ID NO: 128 - RNA, 21 nt, Bos taurus
uacaguacugugauaacugaa

SEQ ID NO: 129 - RNA, 21 nt, Rattus norvegicus
uacaguacugugauaacugaa

SEQ ID NO: 130 - RNA, 21 nt, Rattus norvegicus
uacaguacugugauagcugaa

SEQ ID NO: 131 - RNA, 21 nt, Bos taurus
uaaagugcugacagugcagau

SEQ ID NO: 132 - RNA, 21 nt, Rattus norvegicus
uaaagugcugacagugcagau

SEQ ID NO: 133 - RNA, 22 nt, Homo sapiens
cagugcaaugaugaaagggcau

SEQ ID NO: 134 - RNA, 22 nt, Bos taurus
cagugcaaugaugaaagggcau

SEQ ID NO: 135 - RNA, 22 nt, Rattus norvegicus
cagugcaaugaugaaagggcau

SEQ ID NO: 136 - RNA, 21 nt, Homo sapiens
uaccacaggguagaaccacgg

SEQ ID NO: 137 - RNA, 21 nt, Rattus norvegicus
uaccacaggguagaaccacgg

SEQ ID NO: 138 - RNA, 22 nt, Homo sapiens
uaacacugucugguaaagaugg

SEQ ID NO: 139 - RNA, 22 nt, Bos taurus
uaacacugucugguaaagaugg

SEQ ID NO: 140 - RNA, 22 nt, Rattus norvegicus
uaacacugucugguaaagaugg

SEQ ID NO: 141 - RNA, 21 nt, Homo sapiens
ugagaugaagcacuguagcuc

SEQ ID NO: 142 - RNA, 22 nt, Bos taurus
ugagaugaagcacuguagcucg

SEQ ID NO: 143 - RNA, 22 nt, Rattus norvegicus
ugagaugaagcacuguagcuca

SEQ ID NO: 144 - RNA, 22 nt, Homo sapiens
uggagagaaaggcaguuccuga

SEQ ID NO: 145 - RNA, 22 nt, Bos taurus
uggagagaaaggcaguuccuga

SEQ ID NO: 146 - RNA, 22 nt, Rattus norvegicus
uggagagaaaggcaguuccuga

SEQ ID NO: 147 - RNA, 22 nt, Homo sapiens
caaagaauucuccuuuugggcu

SEQ ID NO: 148 - RNA, 22 nt, Bos taurus
caaagaauucuccuuuugggcu

SEQ ID NO: 149 - RNA, 22 nt, Rattus norvegicus
caaagaauucuccuuuugggcu

SEQ ID NO: 150 - RNA, 21 nt, Homo sapiens
cugaccuaugaauugacagcc

SEQ ID NO: 151 - RNA, 23 nt, Bos taurus
cugaccuaugaauugacagccag

SEQ ID NO: 152 - RNA, 21 nt, Rattus norvegicus
cugaccuaugaauugacagcc

SEQ ID NO: 153 - RNA, 22 nt, Homo sapiens
aacuggccuacaaagucccagu

SEQ ID NO: 154 - RNA, 22 nt, Bos taurus
aacuggccuacaaagucccagu

SEQ ID NO: 155 - RNA, 22 nt, Rattus norvegicus
aacuggccuacaaagucccagu

SEQ ID NO: 156 - RNA, 21 nt, Homo sapiens
uagcagcacagaaauauuggc

SEQ ID NO: 157 - RNA, 22 nt, Bos taurus
uagcagcacagaaauauuggca

SEQ ID NO: 158 - RNA, 21 nt, Rattus norvegicus
uagcagcacagaaauauuggc

SEQ ID NO: 159 - RNA, 22 nt, Homo sapiens
uaacacugucugguaacgaugu

SEQ ID NO: 160 - RNA, 23 nt, Bos taurus
uaacacugucugguaacgauguu

SEQ ID NO: 161 - RNA, 22 nt, Rattus norvegicus
uaacacugucugguaacgaugu

SEQ ID NO: 162 - RNA, 22 nt, Homo sapiens
uccuucauuccaccggagucug

SEQ ID NO: 163 - RNA, 22 nt, Bos taurus
uccuucauuccaccggagucug

SEQ ID NO: 164 - RNA, 22 nt, Rattus norvegicus
uccuucauuccaccggagucug

SEQ ID NO: 165 - RNA, 21 nt, Homo sapiens
uugugcuugaucuaaccaugu

SEQ ID NO: 166 - RNA, 21 nt, Rattus norvegicus
uugugcuugaucuaaccaugu

SEQ ID NO: 167 - RNA, 21 nt, Homo sapiens
ugauuguccaaacgcaauucu

SEQ ID NO: 168 - RNA, 21 nt, Rattus norvegicus
ugauuguccaaacgcaauucu

SEQ ID NO: 169 - RNA, 23 nt, Homo sapiens
agcuacauugucugcuggguuuc

SEQ ID NO: 170 - RNA, 22 nt, Bos taurus
agcuacauugucugcuggguuu

SEQ ID NO: 171 - RNA, 23 nt, Rattus norvegicus
agcuacauugucugcuggguuuc

SEQ ID NO: 172 - RNA, 23 nt, Homo sapiens
cagugcaauaguauugucaaagc

SEQ ID NO: 173 - RNA, 25 nt, Bos taurus
cagugcaauaguauugucaaagcau

SEQ ID NO: 174 - RNA, 23 nt, Rattus norvegicus
cagugcaauaguauugucaaagc

SEQ ID NO: 175 - RNA, 22 nt, Rattus norvegicus
cagcagcaauucauguuuugga

SEQ ID NO: 176 - RNA, 22 nt, Homo sapiens
uuauaaagcaaugagacugauu

SEQ ID NO: 177 - RNA, 23 nt, Bos taurus
uccgucucaguuacuuuauagcc

SEQ ID NO: 178 - RNA, 22 nt, Rattus norvegicus
uuauaaagcaaugagacugauu

SEQ ID NO: 179 - RNA, 22 nt, Homo sapiens
uuaucagaaucuccagggguac

SEQ ID NO: 180 - RNA, 22 nt, Bos taurus
uuaucagaaucuccagggguac

SEQ ID NO: 181 - RNA, 22 nt, Rattus norvegicus
uuaucagaaucuccagggguac

SEQ ID NO: 182 - RNA, 22 nt, Homo sapiens
uaauacugucugguaaaaccgu

SEQ ID NO: 183 - RNA, 22 nt, Bos taurus
uaauacugucugguaaugccgu

SEQ ID NO: 184 - RNA, 22 nt, Rattus norvegicus
uaauacugucugguaaugccgu

SEQ ID NO: 185 - RNA, 22 nt, Homo sapiens
uaugugccuuuggacuacaucg

SEQ ID NO: 186 - RNA, 21 nt, Bos taurus
uaugugccuuuggacuacauc

SEQ ID NO: 187 - RNA, 22 nt, Rattus norvegicus
uaugugccuuuggacuacaucg

SEQ ID NO: 188 - RNA, 22 nt, Rattus norvegicus
uauguguguguguauguccaug

SEQ ID NO: 189 - RNA, 21 nt, Homo sapiens
cagcagcacacugugguuugu

SEQ ID NO: 190 - RNA, 22 nt, Bos taurus
cagcagcacacugugguuugua

SEQ ID NO: 191 - RNA, 22 nt, Rattus norvegicus
cagcagcacacugugguuugua

SEQ ID NO: 192 - RNA, 23 nt, Homo sapiens
uaauccuugcuaccugggugaga

SEQ ID NO: 193 - RNA, 23 nt, Bos taurus
uaauccuugcuaccugggugaga

SEQ ID NO: 194 - RNA, 22 nt, Rattus norvegicus
aaugcaccugggcaaggguuca

SEQ ID NO: 195 - RNA, 23 nt, Homo sapiens
uagcagcgggaacaguucugcag

SEQ ID NO: 196 - RNA, 23 nt, Rattus norvegicus
uagcagcgggaacaguacugcag

SEQ ID NO: 197 - RNA, 22 nt, Homo sapiens
caugccuugaguguaggaccgu

SEQ ID NO: 198 - RNA, 22 nt, Bos taurus
caugccuugaguguaggaccgu

SEQ ID NO: 199 - RNA, 22 nt, Rattus norvegicus
caugccuugaguguaggacugu

SEQ ID NO: 200 - RNA, 22 nt, Homo sapiens
ugugacagauugauaacugaaa

SEQ ID NO: 201 - RNA, 22 nt, Rattus norvegicus
ugugacagauugauaacugaaa

SEQ ID NO: 202 - RNA, 22 nt, Homo sapiens
ugagguaguagguuguauaguu

SEQ ID NO: 203 - RNA, 22 nt, Bos taurus
ugagguaguagguuguauaguu

SEQ ID NO: 204 - RNA, 22 nt, Rattus norvegicus
ugagguaguagguuguauaguu

SEQ ID NO: 205 - RNA, 21 nt, Homo sapiens
cuauacaaucuacugucuuuc

SEQ ID NO: 206 - RNA, 21 nt, Bos taurus
cuauacaaucuacugucuuuc

SEQ ID NO: 207 - RNA, 22 nt, Rattus norvegicus
ugagguaguagguuguauaguu

SEQ ID NO: 208 - RNA, 22 nt, Homo sapiens
ugagguaguagguugugugguu

SEQ ID NO: 209 - RNA, 22 nt, Bos taurus
ugagguaguagguugugugguu

SEQ ID NO: 210 - RNA, 22 nt, Rattus norvegicus
ugagguaguagguugugugguu

SEQ ID NO: 211 - RNA, 22 nt, Homo sapiens
ugagguaguagguuguaugguu

SEQ ID NO: 212 - RNA, 22 nt, Bos taurus
ugagguaguagguuguaugguu

SEQ ID NO: 213 - RNA, 22 nt, Rattus norvegicus
ugagguaguagguuguaugguu

SEQ ID NO: 214 - RNA, 22 nt, Homo sapiens
agagguaguagguugcauaguu

SEQ ID NO: 215 - RNA, 22 nt, Bos taurus
agagguaguagguugcauaguu

SEQ ID NO: 216 - RNA, 22 nt, Rattus norvegicus
agagguaguagguugcauaguu

SEQ ID NO: 217 - RNA, 22 nt, Homo sapiens
ugagguaggagguuguauaguu

SEQ ID NO: 218 - RNA, 21 nt, Bos taurus
ugagguaggagguuguauagu

SEQ ID NO: 219 - RNA, 22 nt, Rattus norvegicus
ugagguaggagguuguauaguu

SEQ ID NO: 220 - RNA, 22 nt, Homo sapiens
ugagguaguagauuguauaguu

SEQ ID NO: 221 - RNA, 22 nt, Bos taurus
ugagguaguagauuguauaguu

SEQ ID NO: 222 - RNA, 22 nt, Rattus norvegicus
ugagguaguagauuguauaguu

SEQ ID NO: 223 - RNA, 22 nt, Rattus norvegicus
ugagguaguaguuugugcuguu

SEQ ID NO: 224 - RNA, 23 nt, Homo sapiens
uacccuguagauccgaauuugug

SEQ ID NO: 225 - RNA, 23 nt, Bos taurus
uacccuguagauccgaauuugug

SEQ ID NO: 226 - RNA, 23 nt, Rattus norvegicus
uacccuguagauccgaauuugug

SEQ ID NO: 227 - RNA, 23 nt, Homo sapiens
uacccuguagaaccgaauuugug

SEQ ID NO: 228 - RNA, 23 nt, Bos taurus
uacccuguagaaccgaauuugug

SEQ ID NO: 229 - RNA, 22 nt, Rattus norvegicus
cccuguagaaccgaauuugugu

SEQ ID NO: 230 - RNA, 22 nt, Homo sapiens
uagcagcacaucaugguuuaca

SEQ ID NO: 231 - RNA, 22 nt, Bos taurus
uagcagcacaucaugguuuaca

SEQ ID NO: 232 - RNA, 22 nt, Rattus norvegicus
uagcagcacaucaugguuuaca

SEQ ID NO: 233 - RNA, 23 nt, Homo sapiens
ugugcaaaucuaugcaaaacuga

SEQ ID NO: 234 - RNA, 23 nt, Bos taurus
ugugcaaaucuaugcaaaacuga

SEQ ID NO: 235 - RNA, 23 nt, Rattus norvegicus
ugugcaaaucuaugcaaaacuga

SEQ ID NO: 236 - RNA, 22 nt, Homo sapiens
acugcauuaugagcacuuaaag

SEQ ID NO: 237 - RNA, 21 nt, Rattus norvegicus
acugcauuacgagcacuuaca

SEQ ID NO: 238 - RNA, 22 nt, Homo sapiens
aagcugccaguugaagaacugu

SEQ ID NO: 239 - RNA, 21 nt, Bos taurus
aagcugccaguugaagaacug

SEQ ID NO: 240 - RNA, 22 nt, Rattus norvegicus
aagcugccaguugaagaacugu

SEQ ID NO: 241 - RNA, 21 nt, Homo sapiens
aucacauugccagggauuacc

SEQ ID NO: 242 - RNA, 21 nt, Rattus norvegicus
aucacauugccagggauuacc

SEQ ID NO: 243 - RNA, 21 nt, Bos taurus
ggguuccuggcaugcugauuu

SEQ ID NO: 244 - RNA, 23 nt, Bos taurus
aucacauugccagggauuaccac

SEQ ID NO: 245 - RNA, 22 nt, Homo sapiens
uggcucaguucagcaggaacag

SEQ ID NO: 246 - RNA, 23 nt, Bos taurus
gugccuacugagcugauaucagu

SEQ ID NO: 247 - RNA, 22 nt, Rattus norvegicus
uggcucaguucagcaggaacag

SEQ ID NO: 248 - RNA, 22 nt, Homo sapiens
cauugcacuugucucggucuga

SEQ ID NO: 249 - RNA, 22 nt, Bos taurus
cauugcacuugucucggucuga

SEQ ID NO: 250 - RNA, 22 nt, Rattus norvegicus
cauugcacuugucucggucuga

SEQ ID NO: 251 - RNA, 22 nt, Homo sapiens
uucaaguaauccaggauaggcu

SEQ ID NO: 252 - RNA, 22 nt, Bos taurus
uucaaguaauccaggauaggcu

SEQ ID NO: 253 - RNA, 22 nt, Rattus norvegicus
uucaaguaauccaggauaggcu

SEQ ID NO: 254 - RNA, 21 nt, Homo sapiens
uucaaguaauucaggauaggu

SEQ ID NO: 255 - RNA, 21 nt, Rattus norvegicus
uucaaguaauucaggauaggu

SEQ ID NO: 256 - RNA, 22 nt, Homo sapiens
aaggagcucacagucuauugag

SEQ ID NO: 257 - RNA, 22 nt, Bos taurus
aaggagcucacagucuauugag

SEQ ID NO: 258 - RNA, 22 nt, Rattus norvegicus
aaggagcucacagucuauugag

SEQ ID NO: 259 - RNA, 22 nt, Homo sapiens
cuuucagucggauguuugcagc

SEQ ID NO: 260 - RNA, 22 nt, Rattus norvegicus
cuuucagucggauguuugcagc

SEQ ID NO: 261 - RNA, 22 nt, Homo sapiens
uguaaacauccuacacucagcu

SEQ ID NO: 262 - RNA, 22 nt, Bos taurus
uguaaacauccuacacucagcu

SEQ ID NO: 263 - RNA, 22 nt, Rattus norvegicus
uguaaacauccuacacucagcu

SEQ ID NO: 264 - RNA, 22 nt, Homo sapiens
cugggagaggguuguuuacucc

SEQ ID NO: 265 - RNA, 22 nt, Rattus norvegicus
cugggagaggguuguuuacucc

SEQ ID NO: 266 - RNA, 22 nt, Homo sapiens
cugggagaaggcuguuuacucu

SEQ ID NO: 267 - RNA, 22 nt, Rattus norvegicus
cugggagaaggcuguuuacucu

SEQ ID NO: 268 - RNA, 22 nt, Homo sapiens
uguaaacauccuugacuggaag

SEQ ID NO: 269 - RNA, 24 nt, Bos taurus
uguaaacauccuugacuggaagcu

SEQ ID NO: 270 - RNA, 22 nt, Rattus norvegicus
uguaaacauccuugacuggaag

SEQ ID NO: 271 - RNA, 21 nt, Homo sapiens
aggcaagaugcuggcauagcu

SEQ ID NO: 272 - RNA, 21 nt, Bos taurus
aggcaagaugcuggcauagcu

SEQ ID NO: 273 - RNA, 22 nt, Rattus norvegicus
aggcaagaugcuggcauagcug

SEQ ID NO: 274 - RNA, 22 nt, Homo sapiens
uggcagugucuuagcugguugu

SEQ ID NO: 275 - RNA, 22 nt, Bos taurus
uggcagugucuuagcugguugu

SEQ ID NO: 276 - RNA, 22 nt, Rattus norvegicus
uggcagugucuuagcugguugu

SEQ ID NO: 277 - RNA, 23 nt, Homo sapiens
uuuggcacuagcacauuuuugcu

SEQ ID NO: 278 - RNA, 23 nt, Bos taurus
uuuggcacuagcacauuuuugcu

SEQ ID NO: 279 - RNA, 23 nt, Rattus norvegicus
uuuggcacuagcacauuuuugcu

SEQ ID NO: 280 - RNA, 22 nt, Homo sapiens
ugagguaguaaguuguauuguu

SEQ ID NO: 281 - RNA, 22 nt, Bos taurus
ugagguaguaaguuguauuguu

SEQ ID NO: 282 - RNA, 22 nt, Rattus norvegicus
ugagguaguaaguuguauuguu

SEQ ID NO: 283 - RNA, 22 nt, Homo sapiens
aacccguagauccgaucuugug

SEQ ID NO: 284 - RNA, 21 nt, Bos taurus
aacccguagauccgaucuugu

SEQ ID NO: 285 - RNA, 22 nt, Rattus norvegicus
aacccguagauccgaucuugug

SEQ ID NO: 286 - RNA, 22 nt, Homo sapiens
cacccguagaaccgaccuugcg

SEQ ID NO: 287 - RNA, 22 nt, Bos taurus
cacccguagaaccgaccuugcg

SEQ ID NO: 288 - RNA, 22 nt, Rattus norvegicus
cacccguagaaccgaccuugcg

SEQ ID NO: 289 - RNA, 23 nt, Homo sapiens
agcagcauuguacagggcuauga

SEQ ID NO: 290 - RNA, 23 nt, Bos taurus
agcagcauuguacagggcuauga

SEQ ID NO: 291 - RNA, 23 nt, Rattus norvegicus
agcagcauuguacagggcuauga

SEQ ID NO: 292 - RNA, 23 nt, Homo sapiens
agcagcauuguacagggcuauca

SEQ ID NO: 293 - RNA, 22 nt, Bos taurus
agcagcauuguacagggcuauc

SEQ ID NO: 294 - RNA, 23 nt, Rattus norvegicus
agcagcauuguacagggcuauca

SEQ ID NO: 295 - RNA, 22 nt, Rattus norvegicus
acaggugagguucuugggagcc

SEQ ID NO: 296 - RNA, 24 nt, Rattus norvegicus
ucccugagacccuuuaaccuguga

SEQ ID NO: 297 - RNA, 22 nt, Rattus norvegicus
ucccugagacccuaacuuguga

SEQ ID NO: 298 - RNA, 22 nt, Homo sapiens
acggguuaggcucuugggagcu

SEQ ID NO: 299 - RNA, 22 nt, Rattus norvegicus
acggguuaggcucuugggagcu

SEQ ID NO: 300 - RNA, 21 nt, Homo sapiens
ucacagugaaccggucucuuu

SEQ ID NO: 301 - RNA, 21 nt, Bos taurus
ucacagugaaccggucucuuu

SEQ ID NO: 302 - RNA, 21 nt, Rattus norvegicus
ucacagugaaccggucucuuu

SEQ ID NO: 303 - RNA, 22 nt, Homo sapiens
cagugcaauguuaaaagggcau

SEQ ID NO: 304 - RNA, 22 nt, Bos taurus
cagugcaauguuaaaagggcau

SEQ ID NO: 305 - RNA, 22 nt, Rattus norvegicus
cagugcaauguuaaaagggcau

SEQ ID NO: 306 - RNA, 22 nt, Homo sapiens
uuugguccccuucaaccagcug

SEQ ID NO: 307 - RNA, 22 nt, Bos taurus
uuugguccccuucaaccagcug

SEQ ID NO: 308 - RNA, 22 nt, Rattus norvegicus
uuugguccccuucaaccagcug

SEQ ID NO: 309 - RNA, 22 nt, Homo sapiens
uuugguccccuucaaccagcua

SEQ ID NO: 310 - RNA, 22 nt, Bos taurus
uuugguccccuucaaccagcua

SEQ ID NO: 311 - RNA, 22 nt, Rattus norvegicus
uuugguccccuucaaccagcua

SEQ ID NO: 312 - RNA, 22 nt, Homo sapiens
ugugacugguugaccagagggg

SEQ ID NO: 313 - RNA, 22 nt, Bos taurus
ugugacugguugaccagagugg

SEQ ID NO: 314 - RNA, 22 nt, Rattus norvegicus
ugugacugguugaccagagggg

SEQ ID NO: 315 - RNA, 22 nt, Homo sapiens
ggagacgcggcccuguuggagu

SEQ ID NO: 316 - RNA, 22 nt, Rattus norvegicus
uggagacgcggcccuguuggag

SEQ ID NO: 317 - RNA, 22 nt, Homo sapiens
cagugguuuuacccuaugguag

SEQ ID NO: 318 - RNA, 22 nt, Bos taurus
uaccacaggguagaaccacgga

SEQ ID NO: 319 - RNA, 22 nt, Rattus norvegicus
cagugguuuuacccuaugguag

SEQ ID NO: 320 - RNA, 24 nt, Rattus norvegicus
ugagaacugaauuccauaggcugu

SEQ ID NO: 321 - RNA, 22 nt, Homo sapiens
ucagugcaucacagaacuuugu

SEQ ID NO: 322 - RNA, 22 nt, Bos taurus
ucagugcaucacagaacuuugu

SEQ ID NO: 323 - RNA, 22 nt, Rattus norvegicus
ucagugcaucacagaacuuugu

SEQ ID NO: 324 - RNA, 21 nt, Homo sapiens
ucgaggagcucacagucuagu

SEQ ID NO: 325 - RNA, 21 nt, Bos taurus
cuagacugaagcuccuugagg

SEQ ID NO: 326 - RNA, 21 nt, Rattus norvegicus
cuagacugaagcuccuugagg

SEQ ID NO: 327 - RNA, 21 nt, Homo sapiens
ucagugcaugacagaacuugg

SEQ ID NO: 328 - RNA, 22 nt, Bos taurus
ucagugcaugacagaacuuggg

SEQ ID NO: 329 - RNA, 21 nt, Rattus norvegicus
ucagugcaugacagaacuugg

SEQ ID NO: 330 - RNA, 23 nt, Rattus norvegicus
aacauucaacgcugucggugagu

SEQ ID NO: 331 - RNA, 22 nt, Homo sapiens
accaucgaccguugauuguacc

SEQ ID NO: 332 - RNA, 22 nt, Rattus norvegicus
accaucgaccguugauuguacc

SEQ ID NO: 333 - RNA, 23 nt, Rattus norvegicus
aacauucauugcugucggugggu

SEQ ID NO: 334 - RNA, 22 nt, Homo sapiens
aacauucaaccugucggugagu

SEQ ID NO: 335 - RNA, 24 nt, Bos taurus
aacauucaaccugucggugaguuu

SEQ ID NO: 336 - RNA, 22 nt, Rattus norvegicus
aacauucaaccugucggugagu

SEQ ID NO: 337 - RNA, 23 nt, Homo sapiens
aacauucauuguugucggugggu

SEQ ID NO: 338 - RNA, 23 nt, Bos taurus
aacauucauuguugucggugggu

SEQ ID NO: 339 - RNA, 23 nt, Rattus norvegicus
aacauucauuguugucggugggu

SEQ ID NO: 340 - RNA, 24 nt, Homo sapiens
uuuggcaaugguagaacucacacu

SEQ ID NO: 341 - RNA, 24 nt, Bos taurus
uuuggcaaugguagaacucacacu

SEQ ID NO: 342 - RNA, 25 nt, Rattus norvegicus
uuuggcaaugguagaacucacaccg

SEQ ID NO: 343 - RNA, 22 nt, Homo sapiens
uauggcacugguagaauucacu

SEQ ID NO: 344 - RNA, 23 nt, Bos taurus
uauggcacugguagaauucacug

SEQ ID NO: 345 - RNA, 22 nt, Rattus norvegicus
uauggcacugguagaauucacu

SEQ ID NO: 346 - RNA, 21 nt, Homo sapiens
caucccuugcaugguggaggg

SEQ ID NO: 347 - RNA, 22 nt, Bos taurus
caucccuugcaugguggagggu

SEQ ID NO: 348 - RNA, 21 nt, Rattus norvegicus
caucccuugcaugguggaggg

SEQ ID NO: 349 - RNA, 22 nt, Rattus norvegicus
uagguaguuucguguuguuggg

SEQ ID NO: 350 - RNA, 22 nt, Homo sapiens
acaguagucugcacauugguua

SEQ ID NO: 351 - RNA, 22 nt, Bos taurus
acaguagucugcacauugguua

SEQ ID NO: 352 - RNA, 22 nt, Rattus norvegicus
acaguagucugcacauugguua

SEQ ID NO: 353 - RNA, 22 nt, Homo sapiens
uaauacugccugguaaugauga

SEQ ID NO: 354 - RNA, 21 nt, Bos taurus
uaauacugccugguaaugaug

SEQ ID NO: 355 - RNA, 23 nt, Rattus norvegicus
uaauacugccugguaaugaugac

SEQ ID NO: 356 - RNA, 23 nt, Homo sapiens
uaauacugccggguaaugaugga

SEQ ID NO: 357 - RNA, 23 nt, Bos taurus
uaauacugccggguaaugaugga

SEQ ID NO: 358 - RNA, 22 nt, Rattus norvegicus
uaauacugccggguaaugaugg

SEQ ID NO: 359 - RNA, 22 nt, Homo sapiens
gugaaauguuuaggaccacuag

SEQ ID NO: 360 - RNA, 22 nt, Rattus norvegicus
gugaaauguuuaggaccacuag

SEQ ID NO: 361 - RNA, 22 nt, Homo sapiens
uucccuuugucauccuaugccu

SEQ ID NO: 362 - RNA, 22 nt, Bos taurus
uucccuuugucauccuaugccu

SEQ ID NO: 363 - RNA, 22 nt, Rattus norvegicus
uucccuuugucauccuaugccu

SEQ ID NO: 364 - RNA, 22 nt, Homo sapiens
uggaauguaaggaagugugugg

SEQ ID NO: 365 - RNA, 22 nt, Bos taurus
uggaauguaaggaagugugugg

SEQ ID NO: 366 - RNA, 22 nt, Rattus norvegicus
uggaauguaaggaagugugugg

SEQ ID NO: 367 - RNA, 22 nt, Homo sapiens
cugugcgugugacagcggcuga

SEQ ID NO: 368 - RNA, 23 nt, Bos taurus
acugugcgugugacagcggcuga

SEQ ID NO: 369 - RNA, 22 nt, Rattus norvegicus
cugugcgugugacagcggcuga

SEQ ID NO: 370 - RNA, 21 nt, Homo sapiens
uaacagucuccagucacggcc

SEQ ID NO: 371 - RNA, 23 nt, Bos taurus
accuuggcucuagacugcuuacu

SEQ ID NO: 372 - RNA, 22 nt, Rattus norvegicus
uaacagucuccagucacggcca

SEQ ID NO: 373 - RNA, 22 nt, Homo sapiens
acagcaggcacagacaggcagu

SEQ ID NO: 374 - RNA, 22 nt, Bos taurus
acagcaggcacagacaggcagu

SEQ ID NO: 375 - RNA, 21 nt, Rattus norvegicus
acagcaggcacagacaggcag

SEQ ID NO: 376 - RNA, 21 nt, Homo sapiens
agcuacaucuggcuacugggu

SEQ ID NO: 377 - RNA, 21 nt, Bos taurus
agcuacaucuggcuacugggu

SEQ ID NO: 378 - RNA, 21 nt, Rattus norvegicus
agcuacaucuggcuacugggu

SEQ ID NO: 379 - RNA, 21 nt, Rattus norvegicus
ugucaguuugucaaauacccc

SEQ ID NO: 380 - RNA, 23 nt, Rattus norvegicus
cucaaacuaugggggcacuuuuu

SEQ ID NO: 381 - RNA, 22 nt, Rattus norvegicus
caucaaaguggaggcccucucu

SEQ ID NO: 382 - RNA, 22 nt, Rattus norvegicus
acucaaacugggggcucuuuug

SEQ ID NO: 383 - RNA, 21 nt, Rattus norvegicus
cucaaaauggaggcccuaucu

SEQ ID NO: 384 - RNA, 21 nt, Homo sapiens
agggcccccccucaauccugu

SEQ ID NO: 385 - RNA, 21 nt, Rattus norvegicus
agggcccccccucaauccugu

SEQ ID NO: 386 - RNA, 22 nt, Homo sapiens
aaaagcuggguugagagggcga

SEQ ID NO: 387 - RNA, 22 nt, Bos taurus
aaaagcuggguugagagggcga

SEQ ID NO: 388 - RNA, 22 nt, Rattus norvegicus
aaaagcuggguugagagggcga

SEQ ID NO: 389 - RNA, 20 nt, Homo sapiens
acugccccaggugcugcugg

SEQ ID NO: 390 - RNA, 22 nt, Rattus norvegicus
ccacugccccaggugcugcugg

SEQ ID NO: 391 - RNA, 23 nt, Homo sapiens
cgcauccccuagggcauuggugu

SEQ ID NO: 392 - RNA, 23 nt, Bos taurus
cgcauccccuagggcauuggugu

SEQ ID NO: 393 - RNA, 23 nt, Rattus norvegicus
cgcauccccuagggcauuggugu

SEQ ID NO: 394 - RNA, 19 nt, Rattus norvegicus
ccuugaggggcaugagggu

SEQ ID NO: 395 - RNA, 22 nt, Homo sapiens
cuggcccucucugcccuuccgu

SEQ ID NO: 396 - RNA, 22 nt, Bos taurus
cuggcccucucugcccuuccgu

SEQ ID NO: 397 - RNA, 22 nt, Rattus norvegicus
cuggcccucucugcccuuccgu

SEQ ID NO: 398 - RNA, 21 nt, Homo sapiens
gccccugggccuauccuagaa

SEQ ID NO: 399 - RNA, 21 nt, Bos taurus
gccccugggccuauccuagaa

SEQ ID NO: 400 - RNA, 21 nt, Rattus norvegicus
gccccugggccuauccuagaa

SEQ ID NO: 401 - RNA, 22 nt, Homo sapiens
acucggcguggcgucggucgug

SEQ ID NO: 402 - RNA, 22 nt, Bos taurus
acucggcguggcgucggucgug

SEQ ID NO: 403 - RNA, 23 nt, Rattus norvegicus
uccgucucaguuacuuuauagcc

SEQ ID NO: 404 - RNA, 21 nt, Rattus norvegicus
ucggucgaucggucggucggu

SEQ ID NO: 405 - RNA, 23 nt, Homo sapiens
ucucacacagaaaucgcacccgu

SEQ ID NO: 406 - RNA, 25 nt, Bos taurus
ucucacacagaaaucgcacccaucu

SEQ ID NO: 407 - RNA, 23 nt, Rattus norvegicus
ucucacacagaaaucgcacccgu

SEQ ID NO: 408 - RNA, 22 nt, Homo sapiens
gcugacuccuaguccagggcuc

SEQ ID NO: 409 - RNA, 21 nt, Bos taurus
gcugacuccuaguccagugcu

SEQ ID NO: 410 - RNA, 21 nt, Rattus norvegicus
ugcugaccccuaguccagugc

SEQ ID NO: 411 - RNA, 19 nt, Rattus norvegicus
ugucccucugggucgccca

SEQ ID NO: 412 - RNA, 21 nt, Rattus norvegicus
agaguaguagguugcauagua

SEQ ID NO: 413 - RNA, 22 nt, Homo sapiens
uaaugccccuaaaaauccuuau

SEQ ID NO: 414 - RNA, 22 nt, Bos taurus
uaaugccccuaaaaauccuuau

SEQ ID NO: 415 - RNA, 22 nt, Rattus norvegicus
uaaugccccuaaaaauccuuau

SEQ ID NO: 416 - RNA, 22 nt, Homo sapiens
gccugcugggguggaaccuggu

SEQ ID NO: 417 - RNA, 22 nt, Bos taurus
gccugcugggguggaaccuggu

SEQ ID NO: 418 - RNA, 23 nt, Rattus norvegicus
gccugcugggguggaaccugguu

SEQ ID NO: 419 - RNA, 22 nt, Homo sapiens
uuuguucguucggcucgcguga

SEQ ID NO: 420 - RNA, 23 nt, Bos taurus
uuuuguucguucggcucgcguga

SEQ ID NO: 421 - RNA, 22 nt, Rattus norvegicus
uuuguucguucggcucgcguga

SEQ ID NO: 422 - RNA, 21 nt, Homo sapiens
acuggacuuggagucagaagg

SEQ ID NO: 423 - RNA, 22 nt, Bos taurus
acuggacuuggagucagaaggc

SEQ ID NO: 424 - RNA, 21 nt, Rattus norvegicus
acuggacuuggagucagaagg

SEQ ID NO: 425 - RNA, 22 nt, Homo sapiens
cuccugacuccagguccugugu

SEQ ID NO: 426 - RNA, 22 nt, Rattus norvegicus
cuccugacuccagguccugugu

SEQ ID NO: 427 - RNA, 23 nt, Homo sapiens
aaugacacgaucacucccguuga

SEQ ID NO: 428 - RNA, 22 nt, Bos taurus
augacacgaucacucccguuga

SEQ ID NO: 429 - RNA, 23 nt, Rattus norvegicus
aaugacacgaucacucccguuga

SEQ ID NO: 430 - RNA, 22 nt, Rattus norvegicus
uauuuagaacggugcuggugug

SEQ ID NO: 431 - RNA, 21 nt, Homo sapiens
ucacuccucuccucccgucuu

SEQ ID NO: 432 - RNA, 21 nt, Bos taurus
ucacuccucuccucccgucuu

SEQ ID NO: 433 - RNA, 23 nt, Rattus norvegicus
ucacuccuccccucccgucuugu

SEQ ID NO: 434 - RNA, 22 nt, Homo sapiens
ucaggcucaguccccucccgau

SEQ ID NO: 435 - RNA, 22 nt, Bos taurus
ucaggcucaguccccucccgau

SEQ ID NO: 436 - RNA, 22 nt, Rattus norvegicus
ucaggcucaguccccucccgau

SEQ ID NO: 437 - RNA, 22 nt, Homo sapiens
ugaaacauacacgggaaaccuc

SEQ ID NO: 438 - RNA, 22 nt, Bos taurus
ugaaacauacacgggaaaccuc

SEQ ID NO: 439 - RNA, 21 nt, Rattus norvegicus
ugaaacauacacgggaaaccu

SEQ ID NO: 440 - RNA, 23 nt, Homo sapiens
ucggggaucaucaugucacgaga

SEQ ID NO: 441 - RNA, 22 nt, Bos taurus
ucggggaucaucaugucacgag

SEQ ID NO: 442 - RNA, 22 nt, Rattus norvegicus
cucggggaucaucaugucacga

SEQ ID NO: 443 - RNA, 21 nt, Homo sapiens
aauggcgccacuaggguugug

SEQ ID NO: 444 - RNA, 21 nt, Rattus norvegicus
aauggcgccacuaggguugug

SEQ ID NO: 445 - RNA, 23 nt, Homo sapiens
ugagguugguguacuguguguga

SEQ ID NO: 446 - RNA, 23 nt, Rattus norvegicus
ugagguugguguacuguguguga

SEQ ID NO: 447 - RNA, 21 nt, Bos taurus
ucaauggcugaggugagguac

SEQ ID NO: 448 - RNA, 21 nt, Rattus norvegicus
ucaauggcugaggugaggcac

SEQ ID NO: 449 - RNA, 20 nt, Homo sapiens
cggcucugggucugugggga

SEQ ID NO: 450 - RNA, 22 nt, Bos taurus
ccccucaguccaccagagcccg

SEQ ID NO: 451 - RNA, 20 nt, Rattus norvegicus
cggcucugggucugugggga

SEQ ID NO: 452 - RNA, 21 nt, Homo sapiens
aagguuacuuguuaguucagg

SEQ ID NO: 453 - RNA, 21 nt, Rattus norvegicus
aagguuacuuguuaguucagg

SEQ ID NO: 454 - RNA, 22 nt, Homo sapiens
cugcccuggcccgagggaccga

SEQ ID NO: 455 - RNA, 22 nt, Bos taurus
cugcccuggcccgagggaccga

SEQ ID NO: 456 - RNA, 22 nt, Rattus norvegicus
cugcccuggcccgagggaccga

SEQ ID NO: 457 - RNA, 19 nt, Homo sapiens
gugaggacucgggaggugg

SEQ ID NO: 458 - RNA, 21 nt, Bos taurus
gugaggacucgggagguggag

SEQ ID NO: 459 - RNA, 21 nt, Rattus norvegicus
gugaggacuggggagguggag

SEQ ID NO: 460 - RNA, 22 nt, Rattus norvegicus
ugggucuuugcgggcaagauga

SEQ ID NO: 461 - RNA, 22 nt, Homo sapiens
ugggucuuugcgggcgagauga

SEQ ID NO: 462 - RNA, 22 nt, Bos taurus
ugggucuuugcgggcgagauga

SEQ ID NO: 463 - RNA, 22 nt, Homo sapiens
gaauguugcucggugaaccccu

SEQ ID NO: 464 - RNA, 20 nt, Rattus norvegicus
aauguugcucggugaacccc

SEQ ID NO: 465 - RNA, 23 nt, Bos taurus
agguuacccgagcaacuuugcau

SEQ ID NO: 466 - RNA, 23 nt, Homo sapiens
uauucauuuauccccagccuaca

SEQ ID NO: 467 - RNA, 22 nt, Bos taurus
caggcugggguguguguggaug

SEQ ID NO: 468 - RNA, 22 nt, Rattus norvegicus
uauucauuuacuccccagccua

SEQ ID NO: 469 - RNA, 20 nt, Homo sapiens
guagaggagauggcgcaggg

SEQ ID NO: 470 - RNA, 20 nt, Bos taurus
guagaggagauggcgcaggg

SEQ ID NO: 471 - RNA, 20 nt, Rattus norvegicus
guagaggagauggcgcaggg

SEQ ID NO: 472 - RNA, 22 nt, Bos taurus
uucaaguaauucaggauagguu

SEQ ID NO: 473 - RNA, 22 nt, Homo sapiens
uagcagcacauaaugguuugug

SEQ ID NO: 474 - RNA, 21 nt, Bos taurus
uagcagcacauaaugguuugu

SEQ ID NO: 475 - RNA, 21 nt, Bos taurus
uagcagcacguaaauauuggc

SEQ ID NO: 476 - RNA, 24 nt, Bos taurus
uguaaacacccuacacucucagcu

SEQ ID NO: 477 - RNA, 22 nt, Bos taurus
aaaagugcuuacagugcaggua

SEQ ID NO: 478 - RNA, 22 nt, Homo sapiens
ucguaccgugaguaauaaugcg

SEQ ID NO: 479 - RNA, 21 nt, Bos taurus
cguaccgugaguaauaaugcg

SEQ ID NO: 480 - RNA, 22 nt, Rattus norvegicus
ucguaccgugaguaauaaugcg

SEQ ID NO: 481 - RNA, 22 nt, Homo sapiens
aagcccuuaccccaaaaagcau

SEQ ID NO: 482 - RNA, 22 nt, Bos taurus
aagcccuuaccccaaaaagcau

SEQ ID NO: 483 - RNA, 22 nt, Homo sapiens
uggacggagaacugauaagggu

SEQ ID NO: 484 - RNA, 22 nt, Bos taurus
uggacggagaacugauaagggu

SEQ ID NO: 485 - RNA, 22 nt, Rattus norvegicus
uggacggagaacugauaagggu

SEQ ID NO: 486 - RNA, 22 nt, Homo sapiens
uagguaguuucauguuguuggg

SEQ ID NO: 487 - RNA, 22 nt, Bos taurus
uagguaguuucauguuguuggg

SEQ ID NO: 488 - RNA, 22 nt, Rattus norvegicus
uagguaguuucauguuguuggg

SEQ ID NO: 489 - RNA, 22 nt, Homo sapiens
uccagcaucagugauuuuguug

SEQ ID NO: 490 - RNA, 23 nt, Bos taurus
uccagcaucagugauuuuguuga

SEQ ID NO: 491 - RNA, 23 nt, Rattus norvegicus
uccagcaucagugauuuuguuga

SEQ ID NO: 492 - RNA, 24 nt, Homo sapiens
aauccuuggaaccuaggugugagu

SEQ ID NO: 493 - RNA, 24 nt, Bos taurus
aauccuuggaaccuaggugugagu

SEQ ID NO: 494 - RNA, 24 nt, Rattus norvegicus
aauccuuggaaccuaggugugaau

SEQ ID NO: 495 - RNA, 22 nt, Homo sapiens
aacuguuugcagaggaaacuga

SEQ ID NO: 496 - RNA, 22 nt, Bos taurus
uguuugcagaggaaacugagac

SEQ ID NO: 497 - RNA, 22 nt, Homo sapiens
uccuguacugagcugccccgag

SEQ ID NO: 498 - RNA, 22 nt, Bos taurus
uccuguacugagcugccccgag

SEQ ID NO: 499 - RNA, 22 nt, Homo sapiens
uuaugguuugccugggacugag

SEQ ID NO: 500 - RNA, 19 nt, Bos taurus
ugguuugccugggacugag

SEQ ID NO: 501 - RNA, 23 nt, Homo sapiens
aaggagcuuacaaucuagcuggg

SEQ ID NO: 502 - RNA, 23 nt, Bos taurus
aaggagcuuacaaucuagcuggg

SEQ ID NO: 503 - RNA, 23 nt, Rattus norvegicus
aaggagcuuacaaucuagcuggg

SEQ ID NO: 504 - RNA, 18 nt, Bos taurus
ucgagaaggaggcugcug